(19) United States
(12) Patent Application Publication
(10) Pub. No.: US 2025/0324956 A1
(43) Pub. Date: Oct. 23, 2025

(54) MOBILE TRANSPORT DEVICE FOR TRANSPORTING INSECT LARVAE

(71) Applicant: FARMINSECT GMBH, Bergkirchen (DE)

(72) Inventors: Wolfgang WESTERMEIER, Bergkirchen (DE); Thomas KÜHN, Bergkirchen (DE)

(21) Appl. No.: 18/723,821

(22) PCT Filed: Dec. 21, 2022
(86) PCT No.: PCT/EP2022/087293

§ 371 Date: Jun. 24, 2024

(30) Foreign Application Priority Data
Dec. 22, 2021 (DE) 10 2021 134 208.2

(51) Int. Cl. A01K 67/34; A01K 1/00

(52) U.S. Cl. CPC A01K 67/34; A01K 1/0035; A01K 1/0052; A01K 1/0076

(57) ABSTRACT

A mobile transport device for transporting insect larvae includes a housing having a selectively openable and closable opening and a receiving section within the housing for receiving at least one first insect fattening container. The first insect fattening container accommodates a first insect larvae cohort for fattening, a recirculation fan for partially recirculating air within the housing, an electronic control unit for controlling the recirculation fan, and a first air regulating device having a first ventilation section on a first side and a first exhaust section on a second side. Air enters the first insect fattening container through the first ventilation section and exits the first insect fattening container through the first exhaust section using the recirculation fan.

The invention relates to a mobile transport device for transporting insect larvae.

The increasing demand for protein for livestock farming and for a growing world population requires alternative protein sources. Insect larvae have a high protein content, can be fed with organic waste, and are significantly more climate-friendly to rear than conventional protein sources. Insect larvae are therefore suitable for supplementing or completely replacing protein-rich feed for livestock farming, such as fishmeal.

In addition to providing an alternative source of protein for their livestock, farmers with livestock farms also have the opportunity to utilise their own organic waste by using insect larvae. It therefore makes sense to carry out a large part of the rearing directly on the farmer's premises, so that the farmer benefits from the insect larvae not only in the form of protein-rich feed, but also in the form of natural waste processors.

For this purpose, the insect larvae must be transported from a central reproduction centre to the farmer for further breeding. Devices for transporting insects are already known from the prior art.

Document WO 2019/125162 A1 relates to a device for transporting live insects from a first location to a second location. For this purpose, the transport device comprises a fluid guide element having at least one elongated fluid guide section, a fluid dispensing element, and a feeding device. The newly hatched larvae are introduced into the device and picked up by the laminar fluid flow. Gases such as air, ambient air, ammonia, methane, and nitrogen oxides are specified as possible fluids. The disadvantage of this solution is that only insects with a minimal age difference can be transported. However, it would be desirable if insects with a larger age difference could also be transported.

A transport box for live larvae is known from CN 209684269 U, which can be used for transport over long distances. The transport box comprises a transport body equipped with a plurality of drawer-like larvae storage boxes. The top of the storage boxes has a cover comprising a plurality of ventilation holes. Ventilation windows are disposed on the side walls of the transport body. Each ventilation window comprises a gauze layer and a sealing layer, the sealing layer having two opposing, semi-circular, foldable, serrated partial windows.

WO 2019/053439 A2 further discloses a system for rearing insect larvae, which may be composed of one or more modules for preparing the feed for the larvae and one or more modules for rearing the larvae using the prepared feed. The rearing modules are configured to handle a plurality of trays for receiving or housing larvae and to supply the trays with food. The entire modular system comprises a transportable container, which can take the form of a shipping container. The modules can thus be partially or fully assembled at a first location and delivered to a second location by means of the transportable container. This very complex system is suitable for larger systems, but not for simple and cost-effective transport over shorter distances or shorter periods of time.

Another disadvantage of the previously known transport containers is that it is not possible to transport eggs or larvae safely, and there is no reliable transport process.

The object of the invention is therefore to enable safe transport of insect larvae and/or insect eggs from the central reproduction site to the farmer, wherein the insect larvae have a high probability of survival and can grow. In addition, in embodiments, one aim of the invention is to transport different age groups of insect larvae simultaneously and preferably to meet their different requirements.

The object is achieved by a first aspect of the invention in a mobile transport device of the type mentioned above by a housing having a selectively openable and closable opening, as well as a receiving section disposed inside the housing for receiving at least one first insect fattening container, wherein the first insect fattening container is received in the receiving section and is configured to receive a first insect larvae cohort for fattening. In addition, the mobile transport device according to the first aspect of the invention has a recirculation fan for partially recirculating air within the housing, an electronic control unit preferably for controlling the recirculation fan, and preferably a first air regulating device comprising a first ventilation section on a first side and a first exhaust section on a second side. The first insect fattening container is preferably disposed between the first ventilation section and the first exhaust section and/or comprises said sections. By means of the recirculation fan, air enters the first insect fattening container through the first ventilation section and exits the insect fattening container through the first exhaust section. It is preferable that the first insect larvae cohort is contained in the first insect fattening container. Preferably, the first fattening container is configured to accommodate the first insect larvae cohort and fattening substrate. Preferably, the first fattening container holds a fattening substrate and the first insect larvae cohort.

For optimum climatic conditions for the first insect larvae cohort during transport, it is advantageous to enable an exchange of air within the first insect fattening container in which the first insect larvae cohort is accommodated. According to the first aspect, the air exchange within the first insect fattening container is enabled by air entering the first insect fattening container via the first ventilation section of the first air regulating device and exiting via the first exhaust section of the first air regulating device by means of the recirculation fan. The stale air in the first insect fattening container can thus be replaced with fresh air. In this context, recirculation means that the air circulates within the housing on a path. The path is preferably a closed path. The recirculating air therefore flows from the recirculation fan through the housing and is finally fed back to the recirculation fan.

The first ventilation section and the first exhaust section do not have to be disposed opposite each other, even if this is preferred. They can also be disposed at an angle to each other or offset, for example. However, it is important that they are disposed in such a way that air can enter and exit the first insect fattening container. A direct air flow is not required for this, but may be provided.

The inventors have recognised that such ventilation can reduce the CO2 concentration, humidity, and temperature in the insect fattening container. This has an influence on the growth of the larvae, so that this can be positively influenced.

The first ventilation section preferably comprises a first flow cross-section that can be adjusted by means of a ventilation control unit. The ventilation control unit may be an element of the electronic control unit, for example in the form of a software module, or it may be implemented as an independent unit, which then preferably communicates with the electronic control unit. The first ventilation section and the first exhaust section are preferably disposed on two substantially opposite sides of the first insect fattening container, so that the air can flow through the insect fattening container in a substantially straight line. The first flow cross-section may be adjustable, for example, in that the first air regulating device comprises louvres movably disposed to adjust the first flow cross-section, preferably by means of a first actuator connected to the ventilation control unit. Other adjustment units may also be provided for adjusting the first flow cross-section. Possible options include, for example, two or more perforated panels displaceable relative to each other, rotating diaphragms, iris diaphragms, flaps or similar, as well as combinations thereof.

Preferably, the ventilation control unit is configured to control and adjust the first flow cross-section based on recorded and/or determined data. For example, the ventilation control unit receives data for this purpose from other units of the transport device, such as sensors, control units, and/or radio devices, which obtain data from a cloud service, for example.

Preferably, the ventilation control unit is configured to control the first flow cross-section based on a detected activity of the insect larvae cohort received in the first insect fattening container. Preferably, the ventilation control unit is configured to reduce the first flow cross-section or to set it to a predetermined value if less ventilation of the insect larvae cohort received in the first insect fattening container is required. This may be the case, for example, if the insect larvae cohort produces particularly little CO2, humidity, and/or heat, i.e. has low activity. The specified value may, for example, be stored in a memory and selected depending on the CO2, humidity, and/or heat. Preferably, the ventilation control unit is configured to expand the first flow cross-section or to set it to a predetermined value when greater ventilation of the insect larvae cohort received in the first insect fattening container is required. This may be the case, for example, if the insect larvae cohorts produce a lot of heat, CO2, and/or humidity, i.e. are highly active. The heat produced in the insect fattening container may also be used to heat the air recirculating in the housing. The ventilation control unit is preferably configured to control the first flow cross-section and preferably further flow cross-sections as a function of signals from an activity sensor device and/or an air sensor device described later.

The mobile transport device preferably has a second insect fattening container, which is received in the receiving section and is configured to receive a second insect larvae cohort for fattening. The first and second cohorts of insect larvae may differ, for example, in their age or in their insect larva genus. It is also possible that the first and second insect larvae cohorts originate from the same insect larvae cohort. It is preferable that the second insect larvae cohort is received in the second insect fattening container. Preferably, the second fattening container is configured to receive the second insect larvae cohort and fattening substrate. Preferably, a fattening substrate and the second insect larvae cohort are received in the second fattening container.

In a preferred embodiment, the receiving section has at least one first compartment in which the first insect fattening container is received. It is preferable that the compartment is substantially cuboid in shape. However, other forms of compartmentalisation are also possible. The compartment may be formed like a shelf in a rack. Preferably, the compartment can be opened and closed to receive the first insect fattening container. In this embodiment, the ventilation and exhaust sections are preferably formed or disposed on the side walls of the compartment. In this way, the insect fattening container inside can be designed more simply, for example as a simple box.

Preferably, the first compartment comprises the first air regulating device. It is preferred that the first side having the first ventilation section forms a first side wall of the compartment, and that the second side having the first exhaust section forms a second side wall of the first compartment. Preferably, the first side wall is disposed substantially opposite the second side wall.

The receiving section preferably has a second compartment in which the second insect fattening container is received. Preferably, further compartments are provided, each of which can receive an insect fattening container. The insect fattening containers in the further compartments are preferably also configured to receive further insect larvae cohorts and preferably fattening substrate for fattening. Insect larvae cohorts and fattening substrate are preferably received in the further compartments.

The individual compartments may, for example, be disposed vertically on top of each other, but also horizontally next to each other or both vertically and horizontally in the receiving section. They preferably extend substantially over the entire cross-section of the interior of the mobile transport device.

The second compartment preferably has a second air regulating device. The second air regulating device is preferably designed in the same way as the first air regulating device. The further compartments are also equipped with an air regulating device. This makes it possible to set a separate climate in each compartment that is independent of the further compartments, and the insect larvae cohorts accommodated in the insect fattening containers can be specifically ventilated as a result. Preferably, the first, second, and all further compartments have the same geometric shape.

Preferably, the insect fattening containers are configured to receive insect eggs. It is preferable that the insect eggs are received in the first insect fattening container, in the second insect fattening container, and/or in the further insect fattening containers. The insect eggs may be received in the insect fattening containers in addition to the insect larvae cohorts or as an alternative to the insect larvae cohorts. Preferably, the first insect fattening container, the second insect fattening container, and/or the further insect fattening containers contain insect larvae cohorts, insect eggs, and fattening substrate. In a preferred refinement, the first, the second, and the further insect fattening containers each have one, two, three, or more receiving devices for receiving the insect eggs. It is preferred that the at least one receiving device is disposed in an upper region of the insect fattening container or in a central region of the insect fattening container. Preferably, the receiving device is disposed above the insect fattening container. It is particularly preferable that the receiving device at least partially covers the top of the insect fattening container. The insect eggs are preferably placed on the receiving device. The insect eggs can incubate on the receiving device until the larvae hatch. After hatching, the larvae preferably fall from the receiving device into the insect fattening container. Preferably, the receiving device has openings through which hatched larvae can fall. Preferably, the one or more receiving devices are grilles. The grille preferably has a mesh density that allows freshly hatched insect larvae to pass through.

Preferably, the receiving section divides the interior into a ventilation section and an exhaust section. The ventilation section and the exhaust section are preferably connected in an air-conducting manner via the recirculation fan on the one hand and at least via the first air regulating device on the other. An outlet side of the recirculation fan preferably opens into the ventilation section of the mobile transport device, so that a positive pressure is created in the ventilation section. In the exhaust section, on the other hand, there is preferably a negative pressure. As a result of the pressure difference, a suction effect is created so that the air from the ventilation part flows into the first insect fattening container via the ventilation section of the air regulating device and flows out of the first insect fattening container into the exhaust part via the exhaust section of the air regulating device, thereby achieving an air exchange within the insect fattening container.

In a further preferred embodiment, the first insect fattening container and the second insect fattening container are stackable. In this embodiment, the insect fattening containers can preferably be stacked directly on top of each other. Separate compartments do not have to be provided for this purpose. In this case, the air regulating device may advantageously be implemented directly on the respective insect fattening container in such a way that the first side with the first ventilation section forms a first side wall of the first insect fattening container and the second side with the first exhaust section forms a second side wall of the first insect fattening container. The first side wall and the second side wall are preferably disposed opposite each other, even if they may be at an angle to each other in other embodiments.

The mobile transport device preferably has a storage container or a holder for receiving an air-conditioning material. The storage container comprises a storage container ventilation section on a first side and a storage container exhaust section on a second side. Like the insect fattening container, the storage container is preferably cuboid in shape. It is particularly preferable that the storage container has the same shape as the first, second and all further compartments. For the purposes of the invention, an air-conditioning material comprises a material that can change the condition of the air. The condition variables of air include pressure, temperature, and the amount of substances in the air. Possible changes in the condition of the air are, for example, the heating of air, the cooling of air, the humidification of air, and/or the dehumidification of air.

The storage container ventilation section preferably comprises a storage container flow cross-section that can be adjusted by means of a storage container control unit. It is possible that the storage container control unit is a further element of the electronic control unit, for example in the form of a further software module or part of the ventilation control unit. Alternatively, it may be designed as an independent unit, which then preferably communicates with the electronic control unit and/or the ventilation control unit. Like the first flow cross-section, the storage container flow cross-section may also be adjustable, for example, in that the storage container ventilation section comprises louvres which are movably disposed to adjust the storage container flow cross-section, preferably by means of a storage container actuator connected to the storage container control unit. Further measures for adjusting the storage container flow cross-section may also be provided, such as in particular two perforated panels displaceable relative to each other, rotating diaphragms, iris diaphragms, flaps or the like.

The storage container ventilation section and the storage container exhaust section are preferably disposed on two substantially opposite sides of the storage container. As a result, the air can flow substantially in a straight line through the container and the air-conditioning material contained therein.

Just like the ventilation control unit, the storage container control unit is preferably also configured to adapt the storage container flow cross-section to the climatic conditions within the first, second, and/or further insect fattening container. Preferably, the storage container control unit is configured to reduce the storage container flow cross-section or to set it to a predetermined value if less ventilation of the insect larvae cohorts accommodated in the insect fattening containers is required. This can be the case, for example, if the insect larvae cohort produces particularly little CO2, humidity and/or heat. The specified value can, for example, be stored in a memory and selected depending on CO2, humidity and/or heat values. Preferably, the storage container control unit is configured to increase the storage container flow cross-section or to set it to a predetermined value if higher ventilation of the insect larvae cohorts accommodated in the insect fattening containers is required. This can be the case, for example, if the insect larvae cohorts produce particularly high levels of heat, CO2 and/or humidity.

The air-conditioning material preferably comprises or is one or more of the following materials: Material for air dehumidification, preferably a zeolite material, a material for air cooling, and/or a material for air heating. The inclusion of a material that absorbs CO2 is also conceivable. A possible material for cooling the air is, for example, ice (water), dry ice, nitrogen ice, a ceramic material, a metallic material or another material that cools the air. The material for air cooling is preferably in a solid or liquid state. The material preferably has a cooling surface that transfers cold to the air recirculating inside the housing. The recirculating air can be cooled by means of convection, thermal radiation and/or conduction, for example. In one variant, it is preferable for the material to evaporate for air cooling. Evaporation cools the recirculating air. It is also possible for the air to be cooled to such an extent that the insect larvae in the insect larvae fattening containers freeze or become frozen. It is also possible that an electric heater is provided in the storage container for air heating. The materials are preferably interchangeable and can be replaced according to a cartridge principle.

Part of the invention, in particular a second consideration, which is also claimed independently, is furthermore a mobile transport device for transporting insect larvae, which has a housing with an opening, a receiving section disposed inside the housing for receiving at least a first insect larvae cohort and a cooling unit. With the aid of the cooling unit, it is possible to transport the insect larvae in a cooled, supercooled or partially or completely frozen state, whereby the cooling unit is preferably configured to maintain the mobile transport device at a temperature in a range from 0° C. to 10° C., preferably in a range between 3° C. and 7° C., particularly preferably at 5° C. It should be understood that the mobile transport device according to the second consideration can also be a refinement of the mobile transport device according to the first consideration. In this case, the cooling unit can comprise or be the storage unit for the air-conditioning material or be accommodated in the storage container. The air-conditioning material is preferably a cooling material.

This consideration of the invention is based on the idea that insect larvae can reduce their metabolism when the temperature is lowered and, depending on the temperature, can change into the aggregate state of a solid body, whereby the vitality of the insect larvae is maintained almost indefinitely. This means that the insect larvae fall into a kind of cold torpor in which movement is no longer possible. In this state, the life processes of the insect larvae are reduced to a minimum. This process is reversible, so that the insect larvae can be returned to an active state. The insect larvae can be transported in this frozen and preserved state over a longer period of several days or even weeks without food and can be thawed after transport. After thawing, the insect larvae resume their physiological processes.

The cooling unit can preferably maintain the temperature for a maximum period of 10 days, preferably a maximum of 6 days, more preferably a maximum of 4 days. It is preferable for the cooling unit to have a heat sink with at least one cooling surface as a material that affects the air flow. The cooling unit can transfer the cold to the recirculating air via the cooling surface of the heat sink. The recirculating air can be cooled via the cooling surface by means of convection, thermal radiation, and/or conduction. The cooling unit can be in liquid or solid form. The heat sink can, for example, have the shape of a cuboid, a cone, a plate, or a pellet. It is preferred that the heat sink is and/or comprises an ice (water), a liquid nitrogen (nitrogen ice), a solid CO2 (a dry ice), a cooling compress such as a cool pack, a cooling pad, a Peltier element, a metallic and/or ceramic body or another cooling element. A metallic and/or ceramic body is preferably cooled down to a predetermined temperature before being inserted into the transport device in order to be able to extract heat from the air during transport. In addition to metallic and ceramic bodies, other bodies that are good heat accumulators can also be used, such as a mineral body like a stone or salt.

In one embodiment, the cooling unit is preferably a cooling unit for active cooling or comprises such a cooling unit. The cooling unit for active cooling preferably comprises a fan, a pump and/or a compressor. The cooling unit for active cooling preferably comprises a coolant supply line for conducting coolant and a coolant discharge line for conducting coolant. Preferably, the coolant supply line and the coolant discharge line are connected at least via the fan, the pump or the compressor, with the coolant supply line preferably supplying coolant to the fan, the pump or the compressor and the coolant discharge line preferably discharging coolant from the fan, the pump or the compressor. Preferably, a coolant flows through the cooling unit for active cooling.

The heat sink preferably has a ratio of volume to surface area of less than 25/1, preferably less than 12/1, particularly preferably less than 10/1. A cooling capacity of the cooling unit per day and per 1 kg of insect larvae is preferably in a range from 6 W/1 kg of insect larvae to 9 W/1 kg of insect larvae, preferably in a range from 7 W/1 kg of insect larvae to 8 W/1 kg of insect larvae, particularly preferably in a range from 7.4 W/1 kg of insect larvae to 7.5 W/1 kg of insect larvae. If the heat sink is a dry ice or nitrogen ice, a ratio of dry ice or nitrogen ice to a quantity of insect larvae to be transported per day can preferably be in a range of 1 kg dry ice (nitrogen ice)/2 kg insect larvae and day to 4 kg dry ice (nitrogen ice)/2 kg insect larvae and day, preferably in a range from 3 kg dry ice (nitrogen ice)/2 kg insect larvae and day to 4 kg dry ice (nitrogen ice)/2 kg insect larvae and day, particularly preferably around 3.4 kg dry ice (nitrogen ice)/2 kg insect larvae and day.

In a preferred refinement, the housing has an air inlet section on a first housing section and an air outlet section on a second housing section. The air inlet section and the air outlet section ensure at least partial air exchange between an interior space enclosed by the housing and an environment. The air inlet section preferably opens into the ventilation section and the air outlet section preferably opens into the ventilation section of the interior.

It is also preferable that the mobile transport device has a fresh air fan for introducing air from the environment into the interior enclosed by the housing. The fresh air fan is preferably disposed in or on the air inlet section. The fresh air fan is preferably controlled by the electronic control unit, but can also be controlled by a separate fresh air fan control unit, for example in the form of a further software module of the electronic control unit or as an independent control unit.

The mobile transport device also has an exhaust fan for discharging air from the interior enclosed by the housing into the environment. The exhaust fan is preferably disposed in or on the air outlet section. The exhaust air fan is preferably controlled by the electronic control unit, but can also be controlled by a separate exhaust air fan control unit, which is preferably designed as a further software module of the electronic control unit or as an independent control unit.

A heating device for heating the air is preferably disposed inside the housing. Preferably, the heating device has an electric heating coil made of heating wires and heats the introduced and/or recirculating air. Other designs that heat the air by means of thermal convection are also conceivable and preferred. For this purpose, the heating device is preferably disposed in the ventilation section of the mobile transport device. It is preferable that the heating device can be controlled by means of the electronic control unit. It is also possible for the heating device to be controlled via a separate heating device control unit, which may be a further software module of the electronic control unit. The heat requirement of the air circulating within the housing depends in particular on the average volume of the circulating air and the temperature difference between an ambient temperature and a preferred temperature within the mobile transport device.

In a further embodiment of the mobile transport device, it is also possible for a preferably separate and individually controllable heating device to be disposed in front of or on each ventilation section. This allows the air flowing through an insect fattening container to be heated specifically according to the requirements of the insect larvae cohort disposed in the insect fattening container. Preferably, the heating device control unit is configured to control the individual heating devices as a function of a control of the flow cross-sections by the ventilation control unit, a CO2, humidity and/or heat measurement value in the respective insect fattening container.

The housing preferably has thermal insulation to reduce heat transfer between the interior enclosed by the housing and the environment. Thermal insulation can, for example, be a thermal insulation material or a construction material with thermal insulation properties. The mobile transport device can thus be protected against cooling or heating.

In a preferred refinement, the thermal insulation has a heat transfer coefficient of less than 0.75 W/m²K, preferably less than 0.5 W/m²K, 0.3 W/m²K, 0.2 W/m²K, 0.15 W/m²K, 0.1 W/m²K.

The mobile transport device preferably comprises an activity sensor device for determining an activity of the first insect larvae cohort received in the first insect fattening container. In the event that several insect fattening containers are provided, the activity sensor device is also configured to determine the activity of the second and further insect larvae cohorts accommodated in the other insect fattening containers.

The activity sensor device is preferably configured to detect a first insect fattening container temperature measurement value at least at a first insect fattening container temperature measurement point of the first insect fattening container. It is also possible that a second or further insect fattening container temperature measurement values are recorded at a second or further insect fattening container temperature measurement points of the first insect mass container. In the event that several insect fattening containers are provided, the activity sensor device is configured to record the measured insect fattening container temperature values of the other insect fattening containers.

It is further preferred that the activity sensor device is configured to detect a first insect fattening container humidity measurement value at least at a first insect fattening container humidity measurement point of the first insect fattening container. It is also possible for a second or further insect fattening container humidity measurement points of the first insect fattening container to record a second or further insect fattening container humidity measurement values. In the case of several insect fattening containers, the activity sensor device is configured to record insect fattening container humidity readings from the other insect fattening containers.

The mobile transport device also preferably comprises an air sensor device for detecting a condition of the air in the interior and preferably the surroundings.

The air sensor device is preferably configured to detect a first storage container temperature measurement value at least at a first storage container temperature measurement point of the storage container.

The air sensor device is preferably also configured to detect a first interior humidity measurement value at least at a first interior humidity measurement point within the housing. It is also possible for a second or further interior humidity measurement points within the housing to record a second or further interior humidity measurement values.

In addition, the air sensor device is preferably configured to detect a first interior temperature measurement value at least at a first interior temperature measurement point inside the housing. In a further preferred embodiment, it is possible for a second or further interior temperature measuring points within the housing to be detected by means of the air sensor device.

Preferably, the air sensor device is set up to detect a first exterior humidity measurement value at least at a first exterior humidity measurement point outside the housing for determining an air humidity of the ambient air and consequently for determining an air humidity of the air flowing into the interior.

The air sensor device is also preferably configured to detect a first exterior temperature measurement value at least at a first exterior temperature measurement point outside the housing for determining an air temperature of the ambient air and consequently for determining an air temperature of the air flowing into the interior. This can be used, for example, to adjust the temperature in the interior by supplying cooler air. The measured exterior temperature value can also be used to determine how any heating device should be controlled in order to achieve the desired interior temperature.

In addition, the air sensor device is preferably configured to detect a first measured CO2 concentration value of the air circulating inside the housing at least at a first CO2 measuring point. This can also be used to determine how much air needs to be supplied from outside in order to achieve a desired target concentration.

In a preferred refinement, the electronic control unit of the mobile transport device is configured to process at least the first insect fattening container temperature measurement value and at least the first insect fattening container humidity measurement value to determine an activity of the first insect larvae cohort received in the first insect fattening container. In the event that a second or further insect fattening containers are provided in the mobile transport device, the electronic control unit is also configured to determine an activity of the second and/or further insect larvae cohorts accommodated in the second and/or further insect fattening containers. For this purpose, the electronic control unit can process the measured insect fattening container temperature values and the measured insect fattening container humidity values of the second and/or the other insect fattening containers.

In a further preferred embodiment, the electronic control unit is configured to process at least the first storage container temperature measurement value, at least the first interior humidity measurement value and/or at least the first interior temperature measurement value to determine a condition of the air circulating in the housing.

In a further preferred embodiment, the electronic control unit is configured to process at least the first measured exterior humidity value and at least the first measured exterior temperature value to determine a state of the ambient air.

It is also preferable that the electronic control unit is configured to process at least the detected first CO2 concentration measurement value to determine whether the CO2 concentration measurement value has been exceeded.

In the event that a CO2 concentration measurement value has been determined to be exceeded, the electronic control unit is preferably configured to control the fresh air fan and the exhaust air fan for an air exchange between the interior and the environment. The air exchange enables a defined amount of used air to be released into the environment by means of the exhaust air fan and the same amount of ambient air to be taken into the interior by means of the fresh air fan. As soon as the air exchange is complete, the electronic control unit can control the fresh air fan and the exhaust air fan again and interrupt the air exchange between the interior and the environment. This also prevents air from being constantly released into the environment and heat and humidity from being lost as a result.

It is also preferable that the mobile transport device has an energy storage unit at least for supplying the recirculation fan and the first control unit with electrical energy. Preferably, the energy storage unit also supplies some or all of the other electrical or electronic components with electrical energy. In a preferred embodiment, the mobile transport device has an electrical connection. The mobile transport device can be connected to a power supply via the electrical connection. In this respect, the mobile transport device may be connected to the local power supply at the recipient's premises and operated as a stationary device.

In a preferred refinement, the mobile transport device comprises a unit for remote monitoring. Preferably, the remote monitoring unit is or comprises a radio module. The radio module may be a 4G module, a 5G module, a GSM module or any other radio module. It is preferable that the radio module can send and receive data. Preferably, the radio module sends and receives data to and from a central base station. The central base station is, for example, a central computing unit or a cloud service. The remote monitoring unit is preferably connected to the activity sensor device, which is configured to detect the activity of the insect larvae accommodated in the insect bast containers. The data recorded by the activity sensor device is preferably sent to the central base station via the radio module. The unit for remote monitoring is preferably connected to the air sensor device, which is configured to detect a condition of the air in the interior and preferably the surroundings of the mobile transport device. The data recorded by the air sensor device is preferably sent to the central base station via the radio module. The data recorded by the activity sensor device and the data recorded by the air sensor device are preferably processed at the central base station. In a preferred refinement, the unit for remote monitoring is connected to the electronic control unit. Preferably, the data already processed by the electronic control unit is sent to the central base station. It is preferable for the remote monitoring unit to have a positioning unit, e.g. a GPS tracker. The positioning unit detects the local position of the mobile transport device. The recorded local position can preferably be sent to the central base station via the radio module. It is preferable that the remote monitoring unit receives control commands in the form of data from the central base station. The control commands received are preferably issued to the electronic control unit. The electronic control unit can preferably execute the control commands. Such control commands can include, for example, commands to set the first or further flow cross-sections, commands to heat or cool the air, commands to control the recirculation fan or similar.

In a third consideration, the invention further solves the problem by a method for transporting insect larvae and/or insect eggs with a mobile transport device, comprising the steps of: filling a first insect fattening container with a first insect larvae cohort and/or insect eggs with the addition of fattening substrate, inserting the filled first insect fattening container into a receiving section of the mobile transport device, preferably into a first compartment of the receiving section, transporting the first insect larvae cohort and/or the insect eggs with the mobile transport device from a first location to a second location, and removing the first insect fattening container from the receiving section at the second location. Preferably, the insect larvae cohort is at least partially covered by the fattening substrate.

It is particularly preferable that the fattening substrate to be added comprises a proportion of water. The fattening substrate to be added preferably comprises a proportion of water-binding substances. The fattening substrate to be added preferably includes a proportion of nutrients. The proportion of water in the fattening substrate to be added is preferably in the range of 0% to 90%. A proportion of water-binding substances in the fattening substrate to be added is preferably in the range of 10% to 100%. The proportion of nutrients in the fattening substrate to be added is preferably in the range of 0% to 100%. Water-binding substances are, for example, wheat bran, water-binding gels or other water-binding elements. The consistency of the fattening substrate is preferably porridge-like when added. During the fattening process, the proportion of water in the fattening substrate decreases. As a result, the consistency of the fattening substrate changes and it becomes free-flowing and portionable. The mobile transport device in the method according to the third consideration of the invention is preferably implemented according to a preferred embodiment of the mobile transport device according to the first and/or second consideration of the invention, as described above. In this respect, full reference is made to the description above.

The first site is preferably the central reproduction site of the insect larvae. The insect larvae can first be reproduced here and filled into the first insect fattening container for transport. In the event that a second and/or further insect fattening containers are provided in the mobile transport device, these are also filled with an insect larvae cohort and fattening substrate at the first location. It is preferable that the second and/or the other insect fattening containers are filled with a different insect larvae cohort to the first insect larvae cohort. The second and/or subsequent insect larvae cohorts may differ in age or genus from the first insect larvae cohort. However, the first, second and further insect larval cohorts can also originate from a common insect larval cohort. It is preferred that the second insect fattening container and/or the other insect fattening containers are alternatively or additionally filled with insect eggs. The second location is preferably at the recipient's premises, such as preferably a farmer who uses the insect larvae as feed for his livestock or as a waste processor. After receiving the mobile transport device, the recipient can remove the first insect fattening container from the receiving section and transfer the first insect larvae cohort contained therein to its own rearing device. In the event that several insect fattening containers are filled, the receiver also removes the other insect fattening containers and transfers the insect larvae cohorts contained therein to its own rearing facility.

In a preferred refinement of the method for transporting the insect larvae, the first insect larvae cohort is cooled down to a temperature in a range from 0° C. to 10° C., preferably in a range between 3° C. and 7° C., particularly preferably to 5° C., by means of a cooling unit after it has been picked up in the mobile transport device. The insect larvae reduce their metabolism almost completely. The insect larvae can be transported in this cooled state over a longer period of time without food.

The cooled temperature is maintained inside the mobile transport device during transport by means of the cooling unit, which preferably has a heat sink with a cooling surface through which cold is released to the recirculating air. It is preferred that the heat sink is and/or comprises an ice (water), a liquid nitrogen (nitrogen ice), a solid CO2 (dry ice), a cooling compress such as a cool pack, a cooling pad, a Peltier element, a metallic material, a ceramic material or another cooling element. If the heat sink is or contains dry ice or nitrogen ice, the dry ice or nitrogen ice evaporates as a result of the cooling capacity. Evaporation of the dry ice is preferably in the range of 0.15 kWh/kg to 0.2 kWh/kg, preferably around 0.178 kWh/kg. A cooling capacity of the cooling unit per day and per 1 kg of insect larvae is preferably in a range from 6 W/1 kg of insect larvae to 9 W/1 kg of insect larvae, preferably in a range from 7 W/1 kg of insect larvae to 8 W/1 kg of insect larvae, particularly preferably in a range from 7.4 W/1 kg of insect larvae to 7.5 W/1 kg of insect larvae.

After the insect larvae have been removed from the mobile transport device at the second location, they can be thawed and/or heated and put into an active state. After thawing, the insect larvae resume their physiological processes. However, the insect larvae can also be heated inside the mobile transport box, for example using the heating device. For this purpose, a warm-up programme may be stored in the electronic control unit, which can be executed after the transport has been completed.

The method of transporting insect larvae preferably further comprises the steps of: providing a first insect fattening container temperature signal representing a first insect fattening container temperature reading and providing a first insect fattening container humidity signal representing a first insect fattening container humidity reading from an activity sensor device to an electronic control unit, determining an activity of the first insect larvae cohort received in the first insect fattening container based on the provided insect fattening container temperature signal and the provided insect fattening container humidity signal by means of the electronic control unit, and adjusting control signals from the electronic control unit at least to a recirculation fan based on the determined activity of the first insect larvae cohort received in the first insect fattening container.

As the temperature and humidity increase with increasing activity of the first insect larvae cohort received in the first insect fattening container, a comparatively high activity of the insect larvae can be determined as a result of comparatively high temperature and/or humidity. At comparatively low temperature and/or humidity, a comparatively low activity of the first insect larvae cohort picked up in the first insect fattening container can be determined.

In the event that an activity has been detected that requires an air exchange within the insect fattening container, the recirculation fan is controlled in such a way that an overpressure is created on an outlet side of the recirculation fan, which preferably opens into the ventilation section of the mobile transport device, whereas a negative pressure is created in the ventilation section. As a result of the pressure difference, a suction effect is created so that the air from the ventilation section flows into the first insect fattening container via the ventilation section of the air regulation direction and flows out of the first insect fattening container into the ventilation section via the exhaust section of the air regulating device, thereby enabling an exchange of air within the insect fattening container. In the event that an activity has been detected that requires a comparatively slower or no air exchange in the first insect fattening container, the recirculation fan can be controlled so as to operate at a reduced speed and consequently saves electricity. It is also possible for the recirculation fan to be switched off completely.

The method of transporting insect larvae with the mobile transport device preferably further comprises the step of: adjusting control signals from the electronic control unit to a ventilation control unit on the basis of the determined activity of the first insect larvae cohort accommodated in the first insect fattening container for setting a first flow cross-section on the basis of the determined activity of the first insect larvae cohort accommodated in the first insect larvae container.

The first flow cross-section may be adjustable, for example, in that the first air control device comprises louvres that are movably disposed, preferably by means of a first actuator, to adjust the first flow cross-section. Alternatively, it can also have perforated plates, rotary slides, iris diaphragms, simple flaps or the like that can be moved against each other. The first flow cross-section can therefore be relatively expanded, relatively reduced or completely closed depending on the determined activity of the first insect larvae cohort disposed in the first insect fattening container. This is particularly advantageous in the event that several insect larvae containers are disposed in the mobile transport device, as the flow cross-sections of the insect fattening containers and thus the air flows passing through the respective insect fattening containers can be individually controlled as a result. It is also possible to utilise the heat produced in the insect fattening containers. In this way, an insect fattening container in which the insect larvae cohort being accommodated produces a particularly large amount of heat can be heavily ventilated. For this purpose, the corresponding flow cross-section is relatively expanded. As a result, the recirculating air is heated and the heat can be distributed to the other insect fattening containers.

In a preferred refinement, the method for transporting insect larvae with the mobile transport device comprises the steps of: providing a first storage container temperature signal representing a first storage container temperature reading, providing a first interior humidity signal representing a first interior humidity reading, providing an interior temperature signal representing a first interior temperature reading, and providing a CO2 concentration signal representing the CO2 concentration reading from an air sensor device on the electronic control unit, determining a state of air circulating in the housing on the basis of the provided storage container temperature signal, the provided interior humidity signal and the provided interior temperature signal by means of the electronic control unit and adjusting control signals from the electronic control unit at least to a storage container control unit on the basis of the determined air condition.

The storage container control unit may be used to set a storage container flow cross-section of a storage container ventilation section of a storage container. An air-conditioning material is preferably disposed in the storage container. Like the first flow cross-section, the storage container flow cross-section may be adjustable, for example, by the storage container ventilation section comprising louvres or the other means mentioned, which are preferably disposed movably by means of a storage container actuator to adjust the storage container flow cross-section.

The storage container flow cross-section can therefore be relatively expanded, reduced or completely closed by means of the storage container control unit depending on the detected air condition of the air circulating in the housing. In the case of an at least partially open storage container flow cross-section, the air can flow from the ventilation section into the storage container and flow through the air-conditioned material inside. The air-conditioning material can extract humidity from the air flowing into the storage container as required, add or remove heat, or absorb excess CO2 from the air. It is further preferred that the air-conditioning material cools the incoming air to such an extent that the insect larvae cohorts accommodated in the insect fattening containers no longer show any activity as a result.

It is further preferred that the method comprises the following steps: providing an exterior humidity signal representing a first exterior humidity reading and providing an exterior temperature signal representing a first exterior temperature reading from the air sensor device to the electronic control unit, determining a state of the ambient air based on the provided exterior humidity signal and the provided exterior temperature signal, and adjusting control signals from the electronic control unit at least to a heating device.

In the event that the determined air condition of the ambient air requires a heat supply for the ambient air flowing into the interior, the heating device can be controlled in such a way that it supplies heat to the ambient air flowing into the interior as required. For this purpose, the heating device is preferably disposed on an air inlet section of the housing. The heating device is preferably also set up and disposed so that it can heat the air circulating inside the housing based on the already determined air condition of the air circulating in the housing. The air circulating in the interior is also heated by the waste heat from those insect larvae cohorts that are highly active due to their advanced age. In the event that the determined air condition of the ambient air does not require a heat supply for the ambient air flowing into the interior or for the air recirculating in the housing, the heating device can be switched off.

In the event that a second or further insect fattening containers are disposed in the mobile transport device, it is possible that a separate heating device is disposed in front of each ventilation section of an insect fattening container. The electronic control unit is preferably configured to individually control the individual heating devices based on the determined activity of the insect larvae cohort accommodated in the respective insect fattening container, based on the determined air condition of the air circulating in the housing and/or based on the determined air condition of the ambient air, so that the air flowing into the respective insect fattening container can be individually heated. In the event that the insect larvae cohort of the respective insect fattening container does not require additional heat, the heating device on the corresponding insect fattening container can be switched off.

The method preferably further comprises the steps: providing a CO2 concentration signal representing a measured CO2 concentration value from the air sensor device to the electronic control unit. Determining an excess CO2 concentration measurement value by the electronic control unit in the event that the CO2 concentration value exceeds a predetermined critical CO2 concentration value, adjusting control signals from the electronic control unit to the fresh air fan in the event that an excess CO2 concentration measurement value has been determined, and adjusting control signals from the electronic control unit to the exhaust air fan in the event that an excess CO2 concentration measurement value has been determined.

The critical CO2 concentration value is predetermined and selected in such a way that the survival of the insect larvae cohorts to be transported is not jeopardised. Exceeding the measured CO2 concentration value is recognised if the measured CO2 concentration value exceeds the critical CO2 concentration value by at least 10%, preferably by at least 5%, particularly preferably by less than at least 5%.

If the CO2 concentration exceeds the measured value, both the fresh air fan and the extract air fan are activated to enable air exchange between the interior and the environment. The exhaust fan can preferably discharge a defined amount of used air into the environment. The fresh air fan can preferably be used to introduce the same amount of air into the interior in the form of fresh air from the surroundings. Once the air has been exchanged, the electronic control unit can control the fresh air fan and/or the exhaust air fan again so that no more air is released from the interior of the mobile transport device into the environment or introduced from the environment into the interior. Additionally or alternatively, closures such as flaps or similar can also be provided on the fresh air and/or exhaust air fan in order to close them.

The method of transporting insect larvae with the mobile transport device preferably further comprises the step of: Filling the storage container with an air-conditioning material to influence the condition of the air inside the housing. The air-conditioning material is preferably disposed in the form of a cartridge in the storage container, which makes the air-conditioning material easy to replace. The air-conditioning material preferably comprises or is a material for dehumidifying the air, a material for cooling the air, a material for heating the air and/or a CO2-absorbing material.

Preferably, the process is monitored by means of a remote monitoring unit. The unit for remote monitoring is preferably designed as described above.

In a fourth consideration, the task mentioned at the beginning is also solved by using the mobile transport device. The use of the mobile transport device is preferably to transport insect larvae and/or insect eggs from a first location to a second location, the first location being spatially distant from the second location.

When using the mobile transport device, it is preferable that the transport takes a maximum of 10, preferably a maximum of 6, more preferably a maximum of 4 days.

The mobile transport device according to the use according to the fourth consideration of the invention is preferably designed according to one of the above-described preferred embodiments of a mobile transport device according to the first or second consideration of the invention. In this respect, full reference is made to the description above.

It is understood that the mobile transport device according to the first or second consideration of the invention, the method according to the third consideration of the invention, and the use according to the fourth consideration of the invention comprise identical and similar sub-considerations, particularly as recorded in the dependent claims. In this respect, for preferred embodiments and refinements of one consideration, reference is also made to preferred embodiments and refinements of the other considerations.

Embodiments of the invention are described below using the drawings. The drawings do not necessarily show the embodiments to scale; rather, the drawings are executed in a schematic and/or slightly distorted form wherever useful for clarity. With respect to expansion of the teachings directly discernible from the drawings, reference is made to the applicable prior art. It should be noted that various modifications and changes relating to the form and detail of an embodiment may be made without deviating from the general idea of the invention. The features of the invention disclosed in the description, the drawings, and the claims may be essential, both individually and in any arbitrary combination, for refining the invention. Furthermore, all combinations of two or more of the features disclosed in the description, the drawings, and/or the claims fall within the scope of the invention. The general idea of the invention is not limited to the precise form or the detail of the preferred embodiments shown and described below or limited to a subject-matter that would be limited in comparison with the subject-matter claimed in the claims. Where ranges are given for dimensions, values within the stated limits and the limit values are disclosed and may be used and claimed arbitrarily. For simplicity, the same reference numerals are used below for identical or similar parts, or parts having identical or similar functions.

Further advantages, features, and details of the invention arise from the below description of the preferred embodiments and from the drawings, which show:

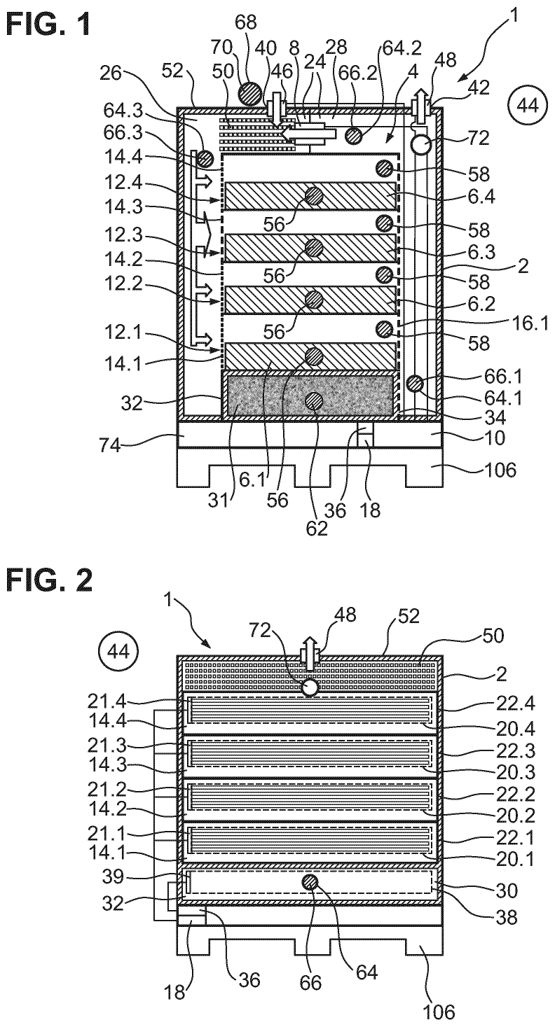

FIG. 1 a section through a first example of the mobile transport device;

FIG. 2 a further section through the mobile transport device according to FIG. 1, perpendicular to the section of FIG. 1;

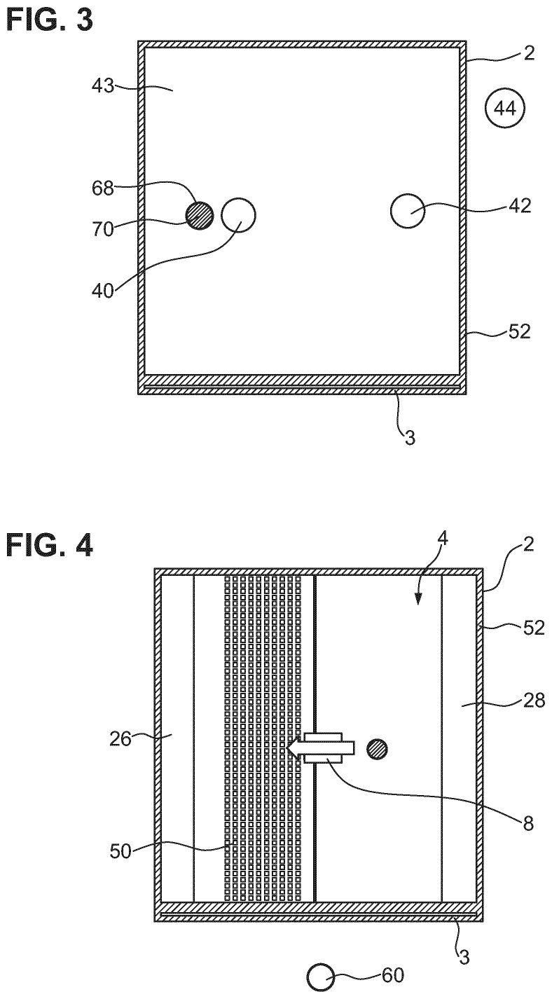

FIG. 3 a top view of the mobile transport device with insulated cover plate of the housing;

FIG. 4 a horizontal section through the mobile transport device;

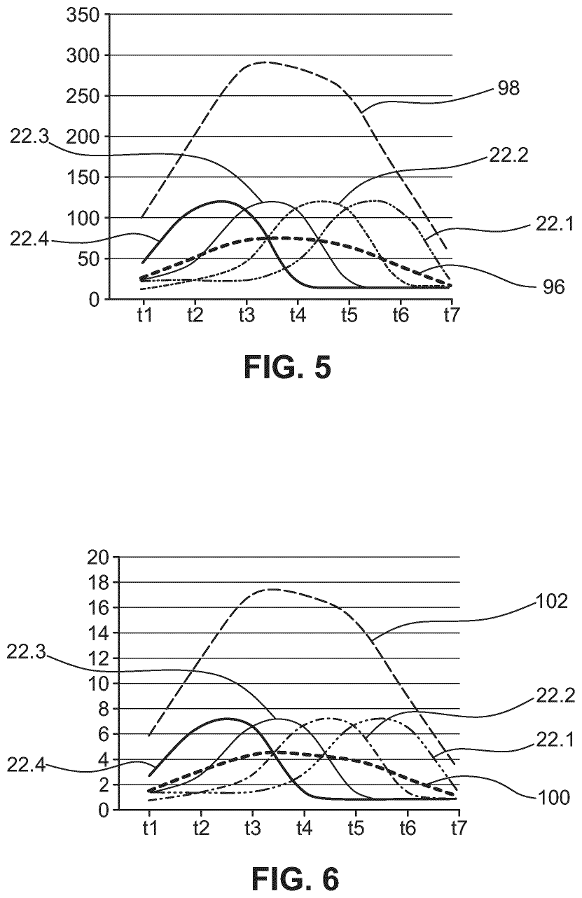

FIG. 5 shows the heat generation of the compartments over time;

FIG. 6 shows the ventilation requirements of the compartments over time;

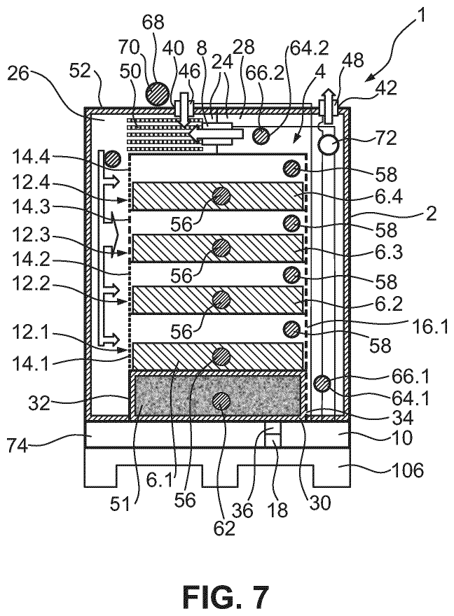

FIG. 7 a section through a second example of the mobile transport device;

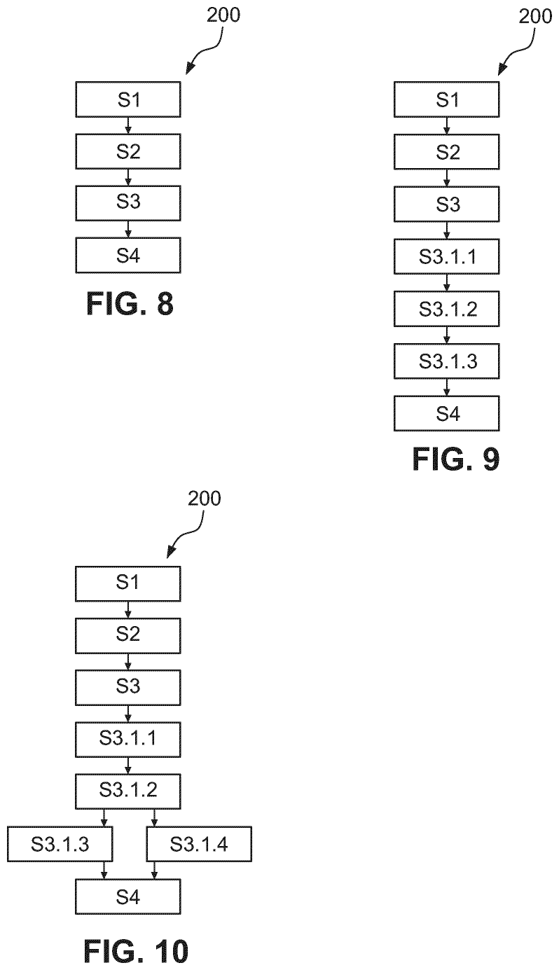

FIG. 8 a schematic flow chart for a first preferred embodiment of the method for transporting insect larvae;

FIG. 9 a schematic flow chart for a second preferred embodiment of the method for transporting insect larvae, which is a possible refinement of the first embodiment of the method for transporting insect larvae;

FIG. 10 a schematic flow chart for a third preferred embodiment of the method for transporting insect larvae, which is a possible refinement of the second embodiment of the method for transporting insect larvae;

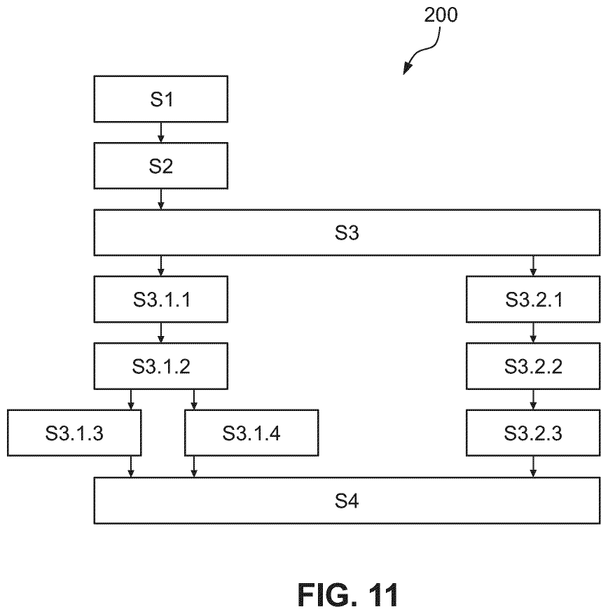

FIG. 11 a schematic flow chart for a fourth preferred embodiment of the method for transporting insect larvae, which is a possible refinement of the first, second or third embodiment of the method for transporting insect larvae;

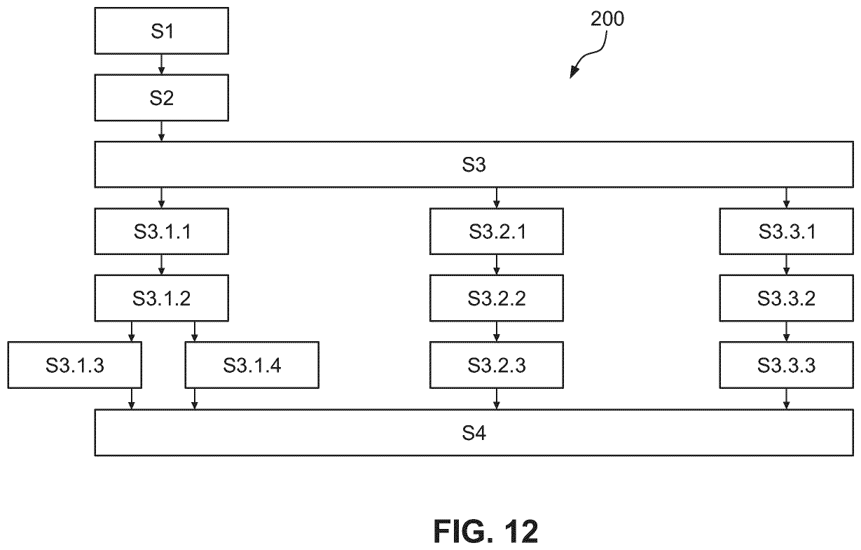

FIG. 12 a schematic flow chart for a fifth preferred embodiment of the method for transporting insect larvae, which is a possible refinement of the first, second, third or fourth embodiment of the method for transporting insect larvae;

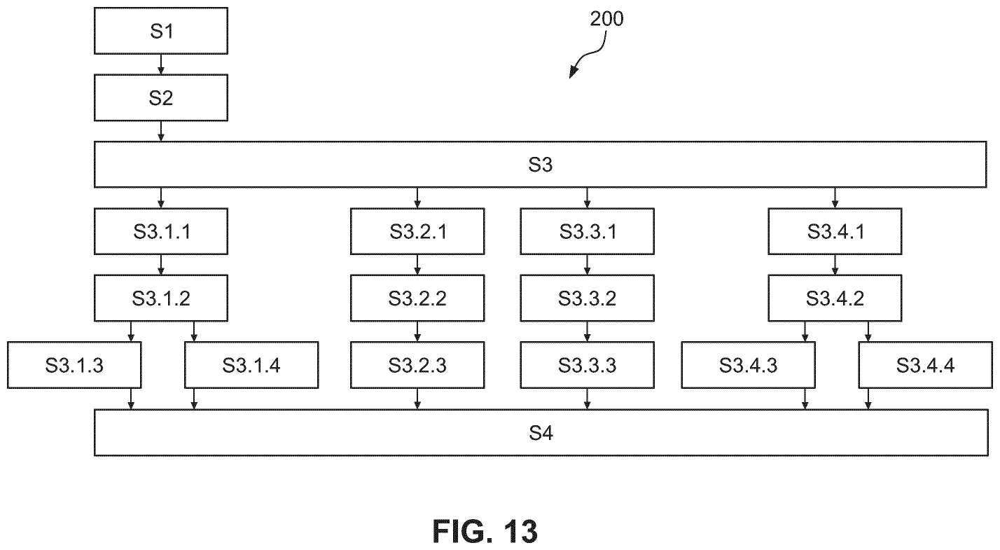

FIG. 13 a schematic flow chart for a sixth preferred embodiment of the method for transporting insect larvae, which is a possible refinement of the first, second, third, fourth or fifth embodiment of the method for transporting insect larvae;

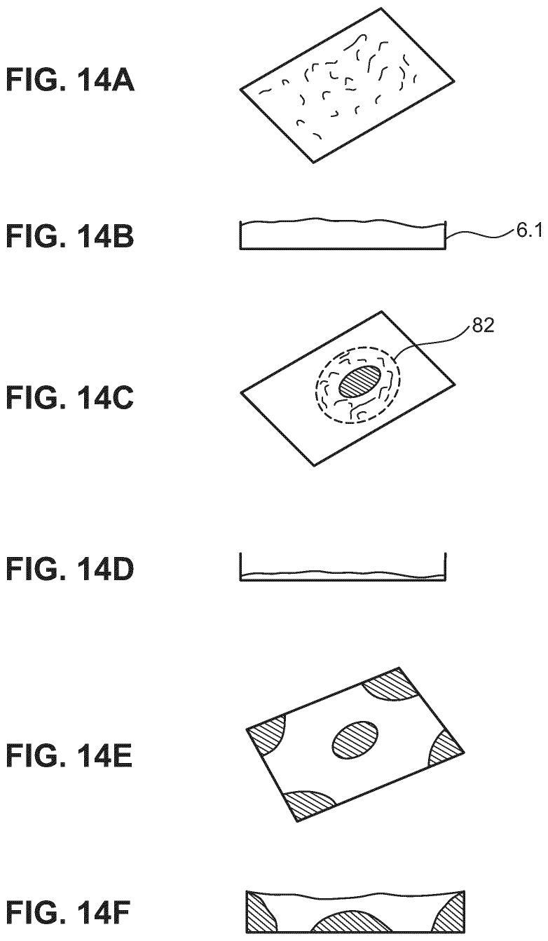

FIG. 14A an isometric top view of a schematic representation of a larval distribution at the beginning of a fattening phase;

FIG. 14B a side view of a schematic representation of the fattening substrate at the beginning of a fattening phase;

FIG. 14C an isometric top view of a schematic representation of a larval distribution in the centre of a fattening phase;

FIG. 14D a side view of a schematic representation of the fattening substrate in the centre of a fattening phase;

FIG. 14E an isometric top view of a schematic representation of a larval distribution at the end of a fattening phase;

FIG. 14F a side view of a schematic representation of a larval distribution at the end of a fattening phase;

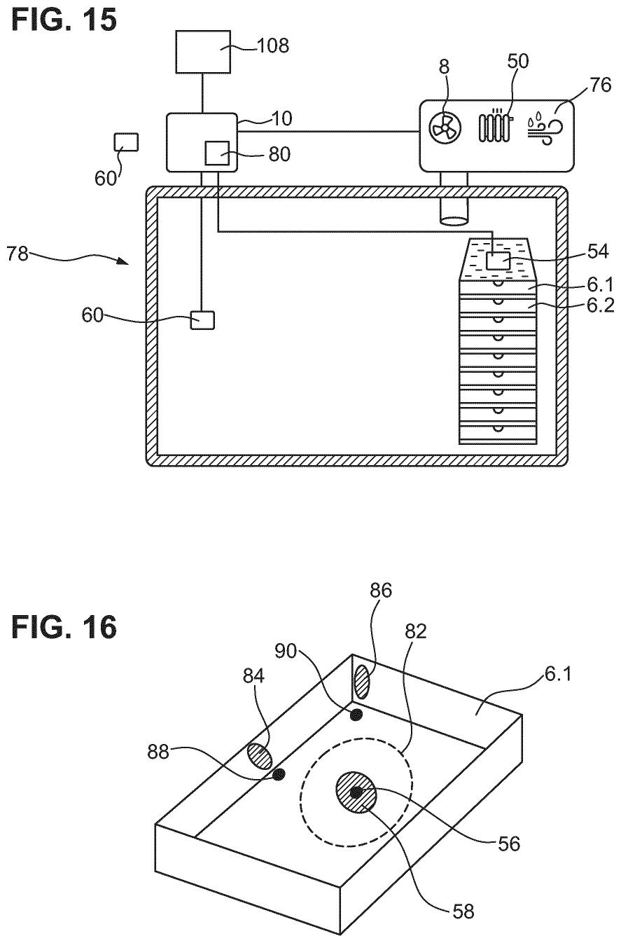

FIG. 15 a schematic view of a stationary insect larvae rearing device;

FIG. 16 an isometric representation of an insect fattening container with activity sensor device for the insect larvae rearing device;

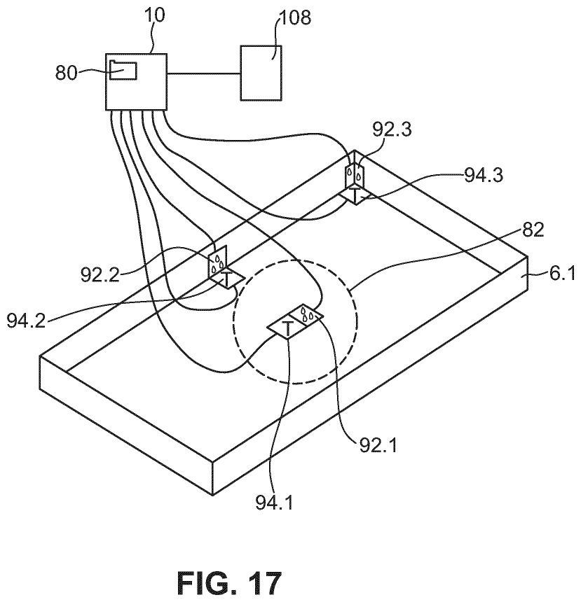

FIG. 17 a further isometric representation of an insect fattening container with activity sensor device for the insect larvae rearing device;

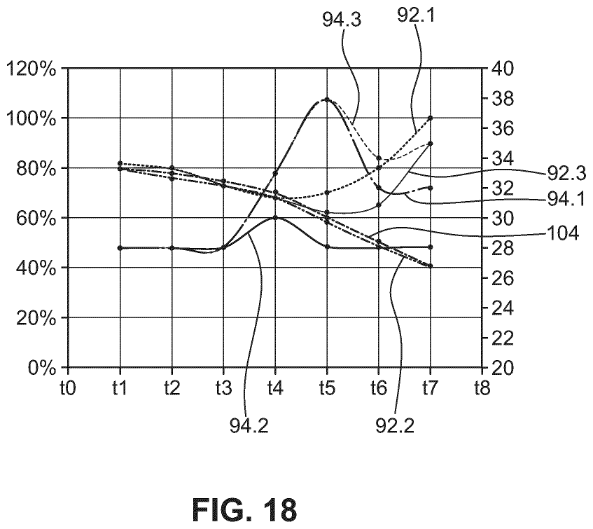

FIG. 18 a time curve of the measured values recorded by the humidity and temperature sensors;

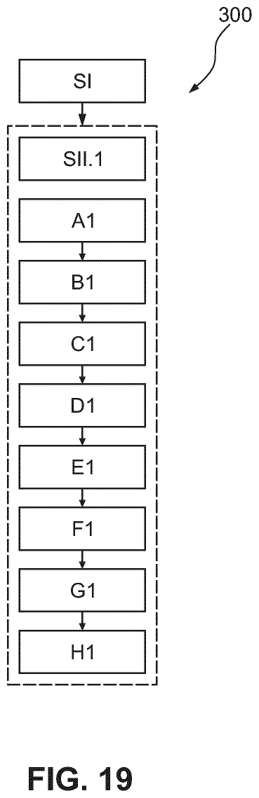

FIG. 19 a schematic flow chart for a first preferred embodiment example of the method for determining the activity of insect larvae;

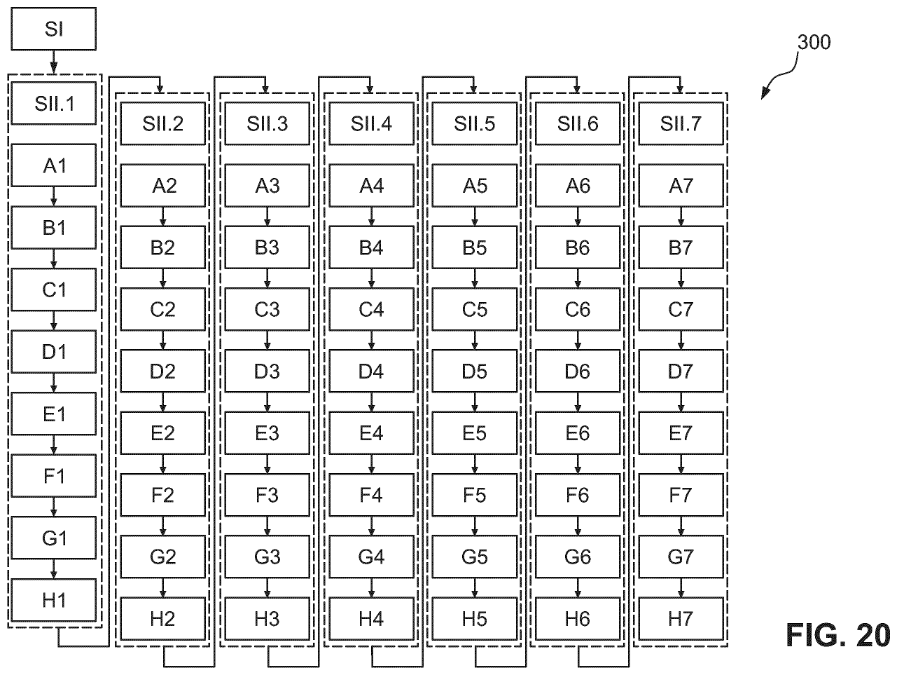

FIG. 20 a schematic flow chart for a second preferred embodiment example of the method for determining an activity of insect larvae, which is a possible refinement of the first embodiment of the method for determining an activity of insect larvae;

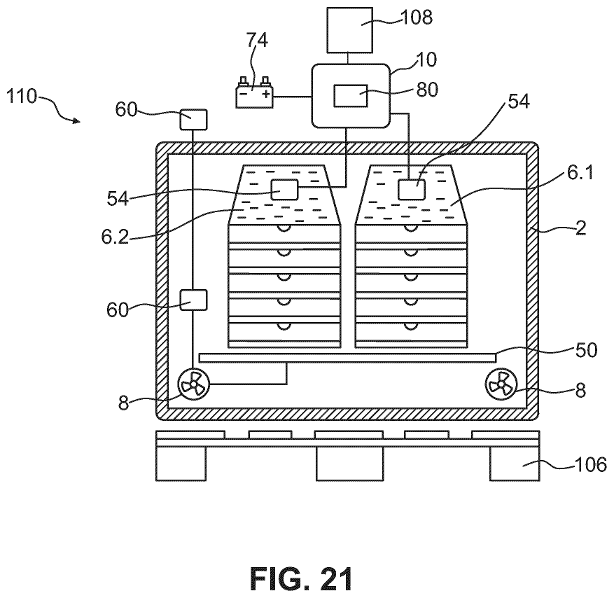

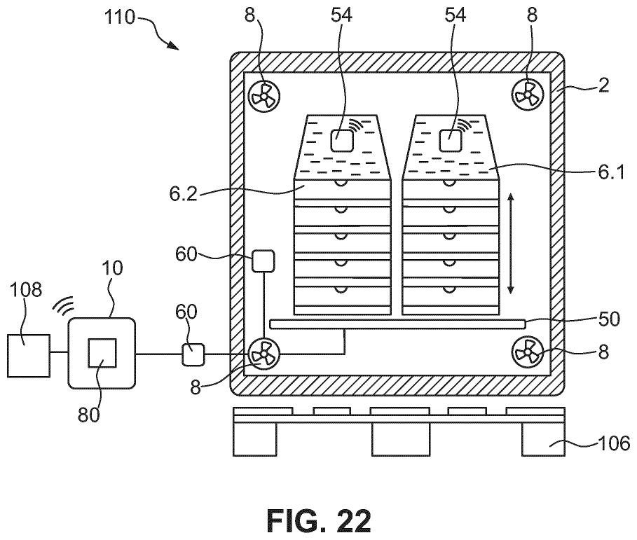

FIG. 21 a second embodiment example of a mobile insect larvae rearing device; and in FIG. 22 a third example of a mobile insect larvae rearing device.

A mobile transport device 1 according to the first consideration of the invention has a housing 2 with a thermal insulation 52, an air inlet section 40, and an air outlet section 42 (FIG. 1). Even though the mobile transport device 1 is described here as mobile, i.e. transportable and intended for transport, its functions and features are also useful in stationary devices for rearing and breeding insect larvae, and it should be understood that these functions and features can also be used to advantage in stationary devices.

A receiving section 4 is provided within the housing, in which four insect fattening containers 6.1-6.4 are disposed in the embodiment example of FIG. 1. The receiving section 4 is divided into four compartments 22.1-22.4 (see FIG. 2) for receiving the four insect fattening containers 6.1-6.4, which are disposed vertically and substantially over the entire cross-section of the mobile transport device 1 (see FIGS. 2 and 4). In this embodiment, the insect fattening containers 6.1-6.4 can be selectively inserted into and removed from the compartments 22.1-22.4. Preferably, the insect fattening containers 6.1-6.4 are filled with insect larvae and fattening substrate before being transported to compartments 22.1-22.4. This may be done manually, for example, by an employee. The fattening area of an insect fattening container 6.1-6.4 is preferably in the range of 0.5 m² to 0.7 m². The fattening substrate, which is added to the insect fattening containers 6.1-6.4 at the beginning, contains a proportion of water. The fattening substrate to be added preferably comprises a proportion of water-binding substances. The fattening substrate to be added preferably comprises a proportion of nutrients. The fattening substrate loses humidity during the fattening process. The insect larvae, the water-binding substances and/or the ventilation/air conditioning remove humidity from the fattening substrate. The consistency of the fattening substrate changes as a result.

After transport, the individual insect fattening containers 6.1-6.4 are then removed from compartments 22.1-22.4. They can then be transported by the recipient to an existing facility at the destination for further rearing and feeding, for example, or harvested directly when mature. The mobile transport device 1 of the invention allows further feeding and rearing even during transport, which can improve the efficiency of breeding and also the quality of the larvae.

The receiving section 4 divides an interior 24 of the housing 2 into an exhaust section 28 and a ventilation section 26, the function of which will be described in more detail below. The four compartments 22.1-22.4 each have an air regulating device 12.1-12.4, wherein the air regulating devices 12.1-12.4 each have a ventilation section 14.1-14.4 and an exhaust section 16.1-16.4. In the embodiment example of FIG. 1, the ventilation sections 14.1-14.4 each form a first side wall of a compartment and the exhaust sections 16.1-16.4 each form a second side wall of a compartment. The first and second side walls are disposed opposite each other. The ventilation sections 14.1-14.4 also comprise flow cross-sections 20.1-20.4 (see FIG. 2), which are adjustable by means of a ventilation control unit 18, which is disposed in a lower section of the mobile transport device 1.

A recirculation fan 8 is disposed in an upper section of the mobile transport device 1 inside the housing 2. During operation, the recirculation fan 8 conveys air from the exhaust section 28 into the ventilation section 26 and thus forms an air-conducting connection between the exhaust section 28 and the ventilation section 26. The first, second, third and fourth air regulating devices 12.1-12.4 form a further air-conducting connection between the ventilation part 26 and the exhaust part 28. The recirculation fan 8 is controlled by an electronic control unit 10 disposed in a lower section of the mobile transport device 1. The recirculation fan 8 is inserted in a partition wall which closes off the entire clear cross-section between an inner wall of the housing 2 and the remaining receiving section 4, so that the ventilation part 26 and the exhaust part 28 are only connected via the recirculation fan 8 on the one hand and the air regulating devices 12.1-12.4 on the other. This ensures that the air conveyed by the recirculation fan 8 actually reaches the individual insect fattening containers 6.1-6.4 to aerate the insect larvae contained therein.

A storage container 30 is also disposed inside the housing 2, which in this embodiment example is also accommodated in the receiving section 4. In other embodiments, it can also be provided at a different location. The storage container 30 is provided together with the four compartments 22.1-22.4 in a vertical arrangement and forms the lower end of the arrangement. In the embodiment example of FIG. 1, the storage container 30 comprises an additional thermal insulation 52. An air-conditioning material 31, such as zeolite for air dehumidification, is reversibly accommodated in the storage container 30.

The storage container 30 has a storage container ventilation section 32 on a first side and a storage container exhaust section 34 on a second side, which is opposite the first side. The storage container ventilation section 32 also comprises a storage container flow cross-section 38, which is adjustable by means of a storage container control unit 36 (see FIG. 2).

The storage container flow cross-section 38 is completely closed in the embodiment example of FIG. 1 and FIG. 2, so that the air from the ventilation section 26 cannot enter the storage container 30. If it is determined that the humidity of the air in the interior is too high, the storage container ventilation section 32 can be partially or fully opened so that air can also circulate through the storage container 30 and thus reduce the humidity of the air. Instead of zeolite as an air-conditioning material 31, other materials are also conceivable, e.g. a cooling material, so that a temperature of the air can be influenced by corresponding actuation of the storage container ventilation section 32 and the recirculation fan 8.

Like the electronic control unit 10 and the ventilation control unit 18, the storage container control unit 36 is disposed in a lower section of the mobile transport device 1. In the embodiment example of FIG. 1, the storage container control unit 36 and the ventilation control unit 18 are provided as separate control units. In other embodiments, these can also be partially or fully integrated into a single electronic control unit, which then performs the function of some or all of the control units. The lower section also contains an energy storage unit 74 for supplying the electrical and electronic components of the mobile transport device 1. The energy store 74 is preferably designed as a rechargeable battery and preferably has a capacity such that electrical and electronic components can be supplied with electrical energy for the entire duration of the transport. It is preferable that the mobile transport device 1 has an electrical connection (not shown) via which the mobile transport device 1 can be connected to a local power supply. The mobile transport device 1 can therefore also be operated in stationary mode without the energy storage unit 74 providing additional energy.

A fresh air fan 46 is disposed in the air inlet section 40 of the housing 2, which ventilates air from an environment 44 into the interior 24. In the embodiment example of FIG. 1, the air inlet section 40 opens into the ventilation section 26 of the interior 24, so that the air from the environment 44 is ventilated into the ventilation section 26. A heating device 50 is disposed in the ventilation section 26, which heats the incoming air. The heating device 50 is also disposed in such a way that the air recirculated by the recirculation fan 8 can be heated at the same time.

An exhaust fan 48 is disposed in the air outlet section 42 of the housing 2, which directs air from the exhaust section 28 of the interior 24 into the environment 44. Both the fresh air fan 46 and the exhaust air fan 48 can be controlled by the electronic control unit 10.

In the embodiment example of FIG. 1, a first insect fattening container temperature measuring point 56 is disposed in each of the four insect fattening containers 6.1-6.4. In each of the compartments 22.1-22.4, in which the insect fattening containers 6.1-6.4 are accommodated, a first insect fattening container humidity measuring point 58 is also provided, which is, as it were, a further insect fattening container temperature measuring point.

A storage container temperature measuring point 62 is disposed in the storage container 30. A first interior humidity measuring point 64.1 and a first interior temperature measuring point 66.1 adjacent to the storage container exhaust section 34, as well as a second interior humidity measuring point 64.2 and a second interior temperature measuring point 66.2 adjacent to the recirculation fan 8 are provided in the exhaust section 28. A CO2 measuring point 72, which is likewise a further interior temperature measuring point, is disposed in the exhaust section 28 adjacent to the exhaust fan 48.

A third interior temperature measuring point 66.3 and a third interior humidity measuring point 64.3 are disposed in the ventilation section 26. An exterior humidity measuring point 68 and an exterior temperature measuring point 70 are disposed outside the housing 2 in the surroundings 44.

All measuring points are connected to the electronic control unit so that it can analyse the corresponding measuring signals from the measuring points.

The mobile transport device 1 is positioned on a pallet 106. This simplifies transport and the mobile transport device 1 can be handled and transported using conventional logistics equipment.

FIG. 2 shows a side view of a further section of the mobile transport device 1, so that the ventilation sections 14.1-14.4 together with the flow cross-sections 20.1-20.4 and the storage container ventilation section together with the storage container flow cross-section 38 can be seen. In the embodiment example of FIG. 2, the flow cross-sections 20.1-20.4 and the storage container flow cross-section 38 comprise discs that can be moved by means of an actuator 21.1-21.4 or a storage container actuator 39. In the embodiment example of FIG. 2, the actuators 21.1-21.4 are controlled by the ventilation control unit 18 and the storage container actuator 39 is controlled by the storage container control unit 36.

The storage container flow cross-section 38 is completely closed. The first, second, and fourth flow cross-sections 20.1, 20.2, 20.4 are partially open so that air from the ventilation section 26 can flow partially into the insect fattening containers 6.1, 6.2, 6.4. The third flow cross-section 20.3, on the other hand, is completely open so that the air can flow into the third insect fattening container 6.3 via the completely open flow cross-section 20.3. As also indicated in FIG. 1 by the arrow in the ventilation section 26, a roughly equal air flow enters the first, second, and fourth compartments 22.1, 22.2, 22.4, and a slightly higher proportion enters the third compartment 22.3. During the breeding phase, the amount of heat produced by the larvae changes, as will be described in more detail. It is typically low at the beginning and then increases after a few days, only to decrease again towards the end of the maturing process. This can be explained in particular by frictional heat caused by the larvae rubbing against each other. As individual compartments 22.1-22.4 can be individually ventilated, the corresponding insect larvae cohort present in the respective compartment 22.1-22.4 can be supplied with an individual and, depending on maturity, adequate air flow in order to be able to optimally adjust the climate in each case.

FIG. 3 shows a top view of the mobile transport device 1 with insulated cover plate 43, which is part of the housing 2. A selectively openable and closable opening 3 is closed in FIG. 3. The air inlet section 40 and the air outlet section 42 are disposed on the insulated cover plate 43. The exterior humidity measuring point 68 and the exterior temperature measuring point 70 are provided in a spatial proximity to the air inlet section 40, so that a humidity and a temperature of the air flowing in via the air inlet section 40 can be detected.

FIG. 4 shows a top view of the mobile transport device without the cover plate of the housing 2. The receiving section 4 divides the interior 24 into an exhaust section 28 and a ventilation section 26. The direction of the arrow indicates that the recirculation fan 8 ventilates the air from the exhaust section 28 into the ventilation section 26, where it can be heated by the heating device 50. A CO2 measuring point 60 and a further exterior temperature measuring point are also disposed in the surroundings, so that a CO2 concentration can be recorded in addition to the humidity and temperature of the incoming air.

FIG. 5 shows curves of heat generation in the different compartments 22.1-22.4 and thus of the insect larvae cohorts recorded therein at different times t1, t2, t3, t4, t5, t6, and t7, which are plotted on the abscissa axis. The points in time represent day 1, day 2, day 3, day 4, day 5, day 6, and day 7 of a joint transport of these compartments 22.1-22.4 by means of the mobile transport device 1. The insect larvae cohorts are of different ages, so that the individual heat generation curves in compartments 22.1-22.4 are shifted.

On the ordinate axis, the heat generation is plotted in watts in a range from 0 to 350 W.

The heat generation within the first compartment 22.1 and thus the first insect larvae cohort accommodated therein is approximately 25 watts at time t1, i.e. on the first day of transport, and remains almost constant until time t3. Heat generation increases from time t3 and reaches a maximum of approx. 120 watts shortly before time t6. The heat generation then drops again to approx. 20 watts by time t7. The heat generation process shows that the insect larvae picked up in the first compartment 22.2 are comparatively young insect larvae at the start of transport.

The heat generation within the second compartment 22.2 and thus the second cohort of insect larvae accommodated therein is approximately 10 watts at time t1, rising to approximately 45 watts by time t3, and then to approximately 120 watts between times t4 and t5. The heat generation then drops to approx. 10 watts by time t6. The heat generation curve shows that the insect larvae picked up in the second compartment 22.2 are comparatively older than the insect larvae picked up in the first compartment 22.1 at the start of transport.

The heat generation within the third compartment 22.3 and thus the third insect larvae cohort accommodated therein is approx. 20 watts at time t1, by time t2 the heat generation already increases to approx. 50 watts and then reaches a maximum of approx. 120 watts between times t3 and t4. The heat generation then drops to approx. 10 watts up to time t5 and remains constant up to time t7. The heat generation curve shows that the insect larvae accommodated in the third compartment 22.3 are comparatively older than the insect larvae accommodated in the first compartment 22.1 and the insect larvae accommodated in the second compartment 22.2 at the start of transport.

Within the fourth compartment 22.4, the heat generation is already approx. 45 watts at time t1. Between the times t2 and t3, heat generation already reaches a maximum of approx. 120 watts. The heat generation then drops to approx. 10 watts up to time t4 and remains constant at approx. 10 watts up to time t7. It can be seen from the progression that the insect larvae accommodated in the fourth compartment 22.4 are the comparatively oldest insect larvae at the beginning of the transport.

Substantially, the curves of the individual compartments therefore show a phase shift of one day.

The heat generated by the insect larvae also produces energy that can be used to heat the circulating air. This can significantly reduce the energy consumption of the energy storage unit 74.

FIG. 5 also shows the course of an average heat generation with recirculation 96, which is achieved via the recirculation fan 8. At time t1, the average heat generation 96 with recirculation is approx. 20 watts; at time t2, it is already 50 watts. Between times t3 and t4, the average heat generation 96 reaches a maximum of approx. 75 watts and then remains almost constant at approx. 75 watts until time t5. The curve of average heat generation with recirculation then flattens out and drops to approx. 10 watts by time t7.

FIG. 5 also shows the course of a sum of the heat generation of compartments 22.1-22.4 without recirculation. The total heat generation without recirculation 98 is just under 100 watts at time t1, 200 watts at time t2 and then a maximum of approx. 290 watts at time t3. The course of the total heat generation without recirculation 98 drops to approx. 260 watts up to time t5, and then to just over 50 watts up to time t7.

The comparison between the average heat generation with recirculation 96 and the total heat generation without recirculation 98 shows that recirculation by means of the recirculation fan 8 results in a lower heat generation in the mobile transport device 1.

FIG. 6 shows curves of the ventilation demand of compartments 22.1-22.4, the average ventilation demand with recirculation 100 and the sum of the ventilation demand of compartments 22.1-22.4 without recirculation 102. On the abscissa axis are the times t1, t2, t3, t4, t5, t6 and t7, where, as in FIG. 5, the times represent day 1, day 2, day 3, day 4, day 5, day 6 and day 7 of the joint transport of these compartments 22.1-22.4 with the mobile transport device 1. On the ordinate axis, the ventilation requirement is listed in m³/h in a range from 0 m³/h to 20 m³/h. The calculated ventilation requirement according to FIG. 6 and the calculated heat generation according to FIG. 5 must be considered together.

The ventilation requirement of the first compartment 22.1 and thus of the insect larvae contained therein is slightly above 1 m³/h at time t1 and remains almost constant at 1 m³/h until time t3. The ventilation requirement of the first compartment 22.1 initially increases to 2 m³/h up to time t4 and then to 7 m³/h between times t5 and t6. The ventilation requirement then drops again to approx. 1 m³/h. The ventilation requirement of the first compartment 22.1 is determined by the heat generation of the first compartment 22.1 as shown in FIG. 6.

The ventilation requirement of the second compartment 22.2 is approx. 0.5 m³/h at time t1 and increases to 2 m³/h by time t3. Between the times t4 and t5, the ventilation requirement of the second compartment 22.2 and thus of the insect larvae accommodated in it reaches a maximum of 7 m³/h. By time t6, the ventilation requirement drops again to approx. 0.5 m³/h and remains constant until time t7. The ventilation requirement of the second compartment 22.2 is determined by the heat generation of the second compartment 22.2 as shown in FIG. 6.

The ventilation requirement of the third compartment 22.3 is just over 1 m³/h at time t1 and increases to 2 m³/h by time t2. A maximum ventilation requirement of 7 m³/h is required between the times t3 and t5. Up to time t7, the ventilation requirement of the third compartment 22.3 and thus of the insect larvae contained therein falls to approx. 0.5 m³/h. The ventilation requirement of the third compartment 22.3 is determined by the heat generation of the third compartment 22.3 as shown in FIG. 6.

The fourth compartment 22.4 already requires a ventilation requirement of over 2 m³/h at time t1. The ventilation requirement already reaches a maximum of 7 m³/h between the times t2 and t3. The ventilation requirement then drops to approx. 0.5 m³/h up to time t4 and remains constant up to time t7. The ventilation requirement of the fourth compartment 22.4 is determined by the heat generation of the fourth compartment 22.4 as shown in FIG. 6.

Here too, the curves of the individual compartments substantially show a phase shift of one day.

The average ventilation requirement of compartments 22.1-22.4 with recirculation 100 is just over 1 m³/h at time t1. Between times t3 and t4, the average ventilation requirement 100 reaches a maximum of just over 4 m³/h and then remains almost constant at around 4 m³/h until time t5. The curve of the average ventilation demand with recirculation 100 then flattens out and drops to approx. 0.5 m³/h by time t7.

The sum of the ventilation requirements of compartments 22.1-22.4 without recirculation 102 is approximately 6 m³/h at time t1, approximately 10 m³/h at time t2 and then a maximum of approximately 17 m³/h at time t3. Up to time t5, the total ventilation requirement without recirculation 102 initially falls to 16 m³/h, then to approx. 3 m³/h up to time t7.

The comparison between the average ventilation requirement with recirculation 100 and the sum of the ventilation requirement without recirculation 102 shows that recirculation by means of the recirculation fan 8 results in a lower ventilation requirement in the mobile transport device 1.

A mobile transport device 1 according to the second embodiment of the invention is shown in FIG. 7. The second embodiment example of the mobile transport device 1 differs from the first embodiment example of the mobile transport device 1 (see FIG. 1) in that a cooling unit 51 is accommodated in the storage container 30. The other features of the second embodiment example of the mobile transport device 1 correspond to the features of the first embodiment example of the mobile transport device 1; identical and similar elements are therefore provided with the same reference signs. In this respect, full reference is made to the description above.

The cooling unit 51 is and/or comprises a cooling body which is an ice (water), a liquid nitrogen (nitrogen ice), a solid CO2 (a dry ice), a cooling compress such as, for example, a cool pack, a cooling pad, a Peltier element, a metallic and/or ceramic and/or mineral material or another cooling element and is configured to cool the insect larvae accommodated in the insect fattening containers 6.1-6.4. The insect larvae can be cooled to such an extent that they are no longer active, i.e. they no longer move. As long as the insect larvae are to be kept in the cooled-down state, the heating device 50 is preferably switched off. By means of the heating device 50, however, it is possible to heat the insect larvae at any time and consequently return them to an active state. The cooling unit can also be a cooling unit for active cooling. The cooling unit for active cooling preferably comprises a fan, a pump or a compressor. The cooling unit for active cooling preferably comprises a coolant supply line for conducting coolant and a coolant discharge line for conducting coolant. Preferably, the coolant supply line and the coolant discharge line are connected at least via the fan, the pump or the compressor, with the coolant supply line preferably supplying coolant to the fan, the pump or the compressor and the coolant discharge line preferably discharging coolant from the fan, the pump or the compressor. Preferably, a coolant flows through the cooling unit for active cooling. The coolant may be a cooling liquid, a gas or another coolant.

At the first insect fattening container temperature measurement point 56, an insect fattening container temperature measurement value can be detected. It can thus be checked whether the temperature in the insect fattening containers 6.1-6.4 is within a range that keeps the insect larvae in the cooled state.

FIG. 8 shows a schematic flow chart for a first preferred embodiment of the method for transporting insect larvae with the mobile transport device 1, comprising filling the first insect fattening container 6.1 with insect larvae with the addition of fattening substrate (step S1), inserting the filled first insect fattening container 6.1 into the receiving section 4 of the mobile transport device 1 (step S2), transporting the insect larvae with the mobile transport device 1 from a first location to a second location (step S3) and removing the first insect fattening container 6.1 from the receiving section 4 at a second location (step S4).

FIG. 9 shows a schematic flow diagram for a second preferred embodiment of the method for transporting insect larvae with the mobile transport device 1, which is a possible refinement of the first embodiment of the method for transporting insect larvae (FIG. 8). During transport (step S3), it comprises providing signals from an activity sensor device 54 to the electronic control unit 10 (step S3.1.1), determining insect larval activity (step S3.1.2) and adjusting control signals from the electronic control unit 10 to the recirculation fan 8 (step S3.1.3) based on the determination in step S3.1.2.

FIG. 10 shows a schematic flow diagram for a third preferred embodiment of the method for transporting insect larvae with the mobile transport device 1, which is a possible refinement of the second embodiment of the method for transporting insect larvae (FIG. 9). In addition to the adjustment in step S3.1.3, it comprises a further adjustment of control signals to the ventilation control unit (step 3.1.4) based on the determination in step S3.1.2.

FIG. 11 shows a schematic flow diagram for a fourth preferred embodiment of the method for transporting insect larvae with the mobile transport device 1, which is a possible refinement of the first, second or third embodiment (FIGS. 8, 9, 10) of the method for transporting insect larvae. During transport in step S3, it comprises providing signals from the air sensor device 60 to the electronic control unit 10 (step S3.2.1), determining an air state of air circulating in the housing 2 (step S3.2.2) and adjusting control signals from the electronic control unit 10 to the storage container control unit 36 (step S3.2.3). Steps S3.1.1, S3.1.2, S3.1.3 and S3.1.4, which are also shown in FIG. 10, are optional.

FIG. 12 shows a schematic flow diagram for a fifth preferred embodiment of the method for transporting insect larvae with the mobile transport device 1, which is a possible refinement of the first, second, third or fourth embodiment (FIGS. 8, 9, 10, 11) of the method for transporting insect larvae. During transport in step S3, it comprises providing signals from the air sensor device 60 to the electronic control unit 10 (step S3.3.1), determining a state of the ambient air (step S3.3.2) and adjusting control signals from the electronic control unit 10 to the heating device 50 (step S3.3.3). Steps S3.1.1, S3.1.2, S3.1.3, S3.1.4, S3.2.1, S3.2.2 and S3.2.3, which are also illustrated in FIG. 10, are optional.

FIG. 13 shows a schematic flow diagram for a sixth preferred embodiment of the method for transporting insect larvae with the mobile transport device 1, which is a possible refinement of the first, second, third, fourth or fifth embodiment (FIGS. 8, 9, 10, 11, 12) of the method for transporting insect larvae. During the transport in step S3, it comprises providing signals from the air sensor device 60 to the electronic control unit 10 (step S3.4.1), determining a CO2 concentration measurement value exceedance (step S3.4.2) and adjusting a control signal from the electronic control unit 10 to the fresh air fan 46 (step S3.4.3) and adjusting control signals from the electronic control unit 10 to the exhaust air fan 48 (step S3.4.4) in the event that a CO2 concentration measurement value exceedance has been determined. Steps S3.1.1, S3.1.2, S3.1.3, S3.1.4, S3.2.1, S3.2.2, S3.2.3, S3.3.1, S3.3.2 and S3.3.3, which are also illustrated in FIG. 10, are optional.

FIGS. 14A-14B show the activity of insect larvae, in particular insect larvae of the black soldier fly, with advancing developmental state of the insect larvae, as known from repeated observation of the insect larvae. At the beginning of a fattening phase, the insect larvae are evenly distributed in the first insect fattening container 6.1 (see FIG. 14A). The insect fattening container is completely filled with fattening substrate (see FIG. 14B).

As development progresses and activity increases, the insect larvae group together in a central section 82 of the insect fattening container 6.1 (see FIG. 14C). The fattening substrate increasingly dries out and at this point substantially only covers the bottom of the insect fattening container 6.1 (see FIG. 14D).

FIGS. 14E and 14F show the distribution of insect larvae at a later stage of development. The insect larvae are now also grouped in the corners of the cuboid insect fattening container 6.1 (see FIG. 14E). The side view as shown in FIG. 14F shows that the insect larvae are not grouped flat on the floor, but are substantially clustered over the entire height of the insect fattening container 6.1.

FIG. 15 shows an optionally stationary insect larvae rearing device 78, which can also be mobile and thus form a mobile transport device 1. All the features described with reference to the insect larvae rearing device 78, in particular with regard to the activity sensor device 54, can also be implemented with reference to the mobile transport device 1.

A first insect fattening container 6.1, a second insect fattening container 6.2 and further insect fattening containers are disposed in the rearing device. The insect fattening containers are stacked vertically in three rows as shown in FIG. 15.

An activity sensor device 54 is provided in each of the insect fattening containers 6.1, 6.2 for detecting the activity of the insect larvae accommodated in the respective insect fattening container 6.1, 6.2. The measured values recorded by the activity sensor device 54 are provided to the electronic control unit 10 and thus to a processing unit 80, which is integrated in the electronic control unit 10. Furthermore, measurement data of an air sensor device 60 is provided at the electronic control unit 10, wherein the air sensor device 60 can detect a state both inside and outside the insect larvae rearing device 78. The electronic control unit 10 is also connected to a computer 108 so that the measured values processed by the processing unit 80 can be displayed for a user.

In addition, a recirculation fan 8, a heating device 50 and a humidifier 76, which can be controlled by the electronic control unit 10, are disposed within the insect larvae rearing device 78.

FIG. 16 shows the arrangement of the activity sensor device 54 within the first insect fattening container 6.1. A first insect fattening container temperature measuring point 56 and a first insect fattening container humidity measuring point 58 are disposed in a central section 82 of the first insect fattening container 6.1. A second insect fattening container temperature measuring point 88 and a second insect fattening container humidity measuring point 84 are disposed in close proximity to one another on a side wall of the first insect fattening container 6.1. A third insect fattening container temperature measuring value 90 and a third insect fattening container humidity measuring value 86 are disposed at a corner of the insect fattening container 6.1. According to FIG. 16, the second and third insect fattening container humidity measurement values 84, 86 also extend over the height of the insect fattening container 6.1.

Based on the observed activity according to FIGS. 14A-14F, it can be assumed that the insect larvae cluster during their development first in the central section 82, i.e. at the first insect fattening container temperature measuring point 56 and at the first insect fattening container humidity measuring point 58, and then additionally at the third insect fattening container temperature measuring point 90 and at the third insect fattening container humidity measuring point 86. However, at the second insect fattening container temperature measuring point 88 and the second insect fattening container humidity measuring point, the insect larvae according to FIGS. 14A-14F will not group together.

FIG. 17 also shows an arrangement of the activity sensor device 54 within the first insect fattening container 6.1, but now with sensors instead of measuring points. A first humidity sensor 92.1 is disposed at the first insect fattening container humidity measuring point 58 (cf. FIG. 16), a second humidity sensor 92.2 is disposed at the second insect fattening container humidity measuring point 84 (cf. FIG. 16) and a third humidity sensor 92.3 is disposed at the third insect fattening container humidity measuring point 86 (cf. FIG. 16).

Furthermore, a first temperature sensor 94.1 is disposed at the first insect fattening container temperature measuring point 56 (cf. FIG. 16), a second temperature sensor 94.2 is disposed at the second insect fattening container temperature measuring point 88 (cf. FIG. 16) and a third temperature sensor 94.3 is disposed at the third insect fattening container temperature measuring point 90 (cf. FIG. 16).

The sensors 92.1-92.4, 94.1-94.4 provide signals representing the detected measured values 56, 58, 84, 86, 88, 90 to the electronic control unit 10 and consequently to the processing unit 80.

FIG. 18 shows curves of the measured values recorded by the humidity and temperature sensors as well as the fattening substrate humidity 104 at the times t0, t1, t2, t3, t4, t5, t6, t7 and t8. The points in time are plotted on the abscissa axis. The left-hand ordinate shows the humidity in percent in a range from 0% to 120%. The temperature in ° C. in a range from 20° C. to 40° C. is plotted on the right-hand ordinate.

The fattening substrate humidity 104, which can be regarded as a reference value for the measured humidity values recorded by the humidity sensors, is 80% at time t1, approximately 70% at time t4 and 40% at time t7. Accordingly, the fattening substrate humidity decreases by 40% between the times t1 and t7.

The first humidity sensor 92.1, which is disposed in the central section 82 (cf. FIG. 17), detects a humidity substantially corresponding to the fattening substrate humidity 104 up to the time t4. From time t4, the humidity detected by the first humidity sensor 92.1 begins to deviate from the fattening substrate humidity 104 and rises to 100% humidity by time t7. It can therefore be assumed that the insect larvae cluster at the first humidity sensor 92.1 from time t4, which means that the measured values recorded represent not only the fattening substrate humidity, but also the additional humidity of the insect larvae.

The second humidity sensor 92.2 with an arrangement according to FIG. 17 detects a humidity that substantially corresponds to the fattening substrate humidity 104. It can therefore be assumed that the insect larvae do not cluster at the second humidity sensor 92.2.

The third humidity sensor 92.3 with an arrangement according to FIG. 17 detects a humidity that substantially corresponds to the fattening substrate humidity 104 up to the time t5. Up to time t6, the humidity initially rises to approx. 65% and then to 90% up to time t7. It can therefore be assumed that the insect larvae cluster at the third humidity sensor 92.3 from time t5, which means that the measured values recorded represent not only the fattening substrate humidity, but also the additional humidity of the insect larvae.

The second temperature sensor 94.2 substantially records a constant temperature of 28° C. over the times t1-t7. Only at time t4 does the second temperature sensor 94.2 detect a temperature of 30° C.

The first temperature sensor 94.1 also detects an substantially constant temperature of approx. 28° C. up to time t3. The recorded temperature then rises to around 33° C. by time t4 and finally to 38° C. by time t5. This rise in temperature is due to an increase in activity and an associated increase in heat emission from the insect larvae, which cluster at the first temperature sensor 94.1. The recorded temperature then drops to approx. 32° C. at times t6 and t7.

The third temperature sensor 94.3 also detects an substantially constant temperature of approx. 28° C. up to time t3. The recorded temperature then rises to around 33° C. by time t4 and finally to 38° C. by time t5. This rise in temperature is due to an increase in activity and an associated increase in heat emission from the insect larvae, which cluster at the third temperature sensor 94.3. The recorded temperature then initially drops to approx. 34° C. at time t6 and then rises slightly to 35° C. by time t7.

FIG. 19 shows a schematic flow chart for a first preferred embodiment of the method for determining an activity of insect larvae with the insect larvae rearing device 78, comprising filling the first insect fattening container 6.1 with insect larvae with the addition of fattening substrate at the beginning of a fattening phase (step SI) and processing the measured values recorded by means of the activity sensor device 54 with the processing unit 80 at a first time t1 (step SII.1). Processing at time t1 in step SII.1 preferably comprises the following steps: comparing the recorded measured values with reference values at time t1 (step A1), determining a reference value shortfall at time t1 (step B1), determining a reference value excess at time t1 (step C1), determining a cluster formation at time t1 (step D1), comparing the cluster formation determined in step D1 with a reference cluster formation at the time t1 (step E1), determining a regular activity of the insect larvae at the time t1 (step F1), determining an irregular activity of the insect larvae at the time t1 (step G1) and outputting a developmental state signal at the time t1 (step H1).

FIG. 20 shows a schematic flow chart for a second preferred embodiment of the method for determining an activity of insect larvae, which is a possible refinement of the first embodiment of the method for determining an activity of insect larvae (FIG. 19).

In this second preferred embodiment method, the processing at time t1 in step SII.1 is followed by processing of the measured values recorded by the activity sensor device 54 with the processing unit 80 at the second time t2 in step SII.2. Processing at time t2 (step SII.2) comprises steps A2-H2, which correspond to steps A1-H1, but are carried out for time t2.

The processing at time t2 in step SII.2 is followed by processing of the measured values recorded by the activity sensor device 54 with the processing unit 80 at the third time t3 in step SII.2. Processing at time t3 (step SII.3) comprises steps A3-H3, which correspond to steps A1-H1 and A2-H2, but are carried out at time t3.

The processing at time t2 in step SII.2 is followed by processing of the measured values recorded by the activity sensor device 54 with the processing unit 80 at a third time t3 in step SII.3. Processing at time t3 (step SII.3) comprises steps A3-H3, which correspond to steps A1-H1 and A2-H2, but are carried out at time t3.

The processing at time t3 in step SII.3 is followed by processing of the measured values recorded by the activity sensor device 54 with the processing unit 80 at a third time t4 in step SII.4. Processing at time t4 (step SII.4) comprises steps A4-H4, which correspond to steps A1-H1, A2-H2 and A3-H3, but are carried out at time t4.

The processing at time t4 in step SII.4 is followed by processing of the measured values recorded by the activity sensor device 54 with the processing unit 80 at a third time t5 in step SII.5. Processing at time t5 (step SII.5) comprises steps A5-H5, which correspond to steps A1-H1, A2-H2, A3-H3 and A4-H4, but are carried out at time t5.

The processing at time t5 in step SII.5 is followed by processing of the measured values recorded by the activity sensor device 54 with the processing unit 80 at a third time t6 in step SII.6. Processing at time t6 (step SII.6) comprises steps A6-H6, which correspond to steps A1-H1, A2-H2, A3-H3. A4-H4 and A5-H5, but are carried out at time t6.

The processing at time t6 in step SII.6 is followed by processing of the measured values recorded by the activity sensor device 54 with the processing unit 80 at a third time t7 in step SII.7. Processing at time t7 (step SII.7) comprises steps A7-H7, which correspond to steps A1-H1, A2-H2, A3-H3, A4-H4, A5-H5, A6-H6, but are carried out at time t7.

FIG. 21 shows an embodiment example of a mobile insect larvae rearing device 110, which can be used as a mobile transport device 1. The embodiment example is based on the embodiment example of the mobile insect transport device 1 and the same and similar elements are provided with the same reference signs as in the first embodiment example. In this respect, full reference is made to the description above. A first insect fattening container 6.1, a second insect fattening container 6.2 and further insect fattening containers are accommodated in the mobile insect larvae rearing device 110. The insect fattening containers are stacked in two rows as shown in FIG. 21. An activity sensor device 54 is provided in each of the insect fattening containers 6.1, 6.2. The measured values recorded by the activity sensor device 54 can be provided to the electronic control unit 10 and to the processing unit 10 integrated therein.

The energy storage unit 74 is connected to the electronic control unit 10 in order to supply it with electrical energy. In the embodiment example according to FIG. 21, a heating device 50 is disposed in a lower section of the mobile insect larvae rearing device 110 in such a way that the insect fattening containers 6.1, 6.2 can be positioned above the heating device 50. In the mobile insect larvae rearing device 110, two recirculation fans 8 are also provided for recirculating air within the mobile insect larvae rearing device 110.

FIG. 22 shows a further embodiment example of the mobile insect larvae rearing device 110, which can be used as a mobile transport device 1. In contrast to the embodiment example according to FIG. 20, four recirculation fans 8 are provided for recirculating air within the mobile insect larvae rearing device 110. In addition, the measured values recorded by the activity sensor device 54 can be provided wirelessly to the electronic control unit 10 and thus wirelessly to the processing unit 80.

1. A mobile transport device for transporting insect larvae, the mobile transport device comprising
a housing having a selectively openable and closeable opening;
a receiving section disposed within the housing, the receiving section for receiving at least one first insect fattening container, wherein the first insect fattening container is configured to receive a first insect larvae cohort for fattening;
a recirculation fan for partial recirculation of air within the housing;
an electronic control unit for controlling the recirculation fan; and
a first air regulating device having a first ventilation section on a first side and a first exhaust section on a second side, and
wherein air enters the first insect fattening container through the first ventilation section and exits the first insect fattening container through the first exhaust section via the recirculation fan.

2. The mobile transport device of claim 1, further comprising a ventilation control unit, wherein the first ventilation section comprises a first flow cross-section adjustable via the ventilation control unit.

3. The mobile transport device of claim 2, wherein the ventilation control unit is configured to control the first flow cross-section based on a determined activity of the first insect larvae cohort received in the first insect fattening container.

4. The mobile transport device of claim 1, further comprising a second insect fattening container received in the receiving section, wherein the second insect fattening container is configured to receive a second insect larvae cohort for fattening.

5. The mobile transport device of claim 4, wherein the receiving section comprises at least one first compartment in which the first insect fattening container is received.

6. The mobile transport device of claim 5, wherein the receiving section comprises at least one second compartment in which the second insect fattening container is received.

7. The mobile transport device of claim 1, wherein the receiving section divides an interior of the housing into a ventilation part and an exhaust part, wherein the ventilation part and the exhaust part are connected in an air-conducting manner via the recirculation fan in one part the one hand and at least via the first air regulating device in the other part and the first air regulating device in the other part.

8. The mobile transport device of claim 1, further comprising a storage container for receiving an air-conditioning material, the storage container comprising a storage container ventilation section on a first side and a storage container exhaust section on a second side, and wherein the storage container ventilation section comprises a storage container flow cross-section adjustable via a storage container control unit.

9. The mobile transport device of claim 8, wherein the air-conditioning material comprises a material for air dehumidification, a material for air cooling, and/or a material for air heating.

10. A mobile transport device for transporting insect larvae, the mobile transport device comprising:
a housing with an opening;
a receiving section disposed within the housing for receiving at least a first insect larvae cohort; and
a cooling unit for cooling of the at least first insect larvae cohort.

11-12. (canceled)

13. The mobile transport device of claim 1, further comprising a fresh air fan for introducing air from the environment into an interior space enclosed by the housing and/or comprising an exhaust air fan for discharging air from the interior space enclosed by the housing into the environment.

14. The mobile transport device of claim 1, further comprising a heating device for heating the air disposed inside the housing.

15. The mobile transport device of claim 1, further comprising an activity sensor device for detecting an activity of the first insect larvae cohort received in the first insect fattening container.

16. The mobile transport device of claim 15, wherein the activity sensor device is configured to detect a first insect fattening container temperature measurement value at least at a first insect fattening container temperature measurement point of the first insect fattening container and/or to detect a first insect fattening container humidity measurement value at a first insect fattening container humidity measurement point of the first insect fattening container.

17. The mobile transport device of claim 13, further comprising an air sensor device for determining a condition of the air in an interior of the housing and/or an environment surrounding the housing.

18. The mobile transport device of claim 17, wherein the air sensor device is configured to detect a first interior humidity measurement value at least at a first interior humidity measurement point within the housing and/or to detect a first interior temperature measurement value at least at a first interior temperature measurement point within the housing.

19. The mobile transport device of claim 17, wherein the air sensor device is configured to detect a first exterior humidity measurement value at a first exterior humidity measurement point outside the housing and/or to detect a first exterior temperature measurement value at least at a first exterior temperature measurement point outside the housing.

20. The mobile transport device of claim 17, wherein;
the air sensor device is configured to detect a first CO2 concentration measurement value of the air circulating within the housing at a first CO2 measurement point,
electronic control unit is configured to process the detected first CO2 concentration measurement value to determine a CO2 concentration measurement value exceedance,
the electronic control unit is configured to control the fresh air fan and the exhaust air fan for an air exchange between the interior space and the environment if a CO2 concentration measurement value exceedance has been determined.

21. The mobile transport device of claim 16, wherein the electronic control unit is configured to process at least the first insect fattening container temperature measurement value and at least the first insect fattening container humidity measurement value for determining an activity of the first insect larvae cohort received in the first insect fattening container.

22. The mobile transport device of claim 18, wherein the electronic control unit is configured to process at least the first interior humidity measurement value and/or at least the first interior temperature measurement value for determining a condition of the air circulating in the housing.

23. The mobile transport device of claim 19, wherein the electronic control unit is configured to process at least the first exterior humidity measurement value and/or at least the first exterior temperature measurement value for determining a condition of the ambient air.

24. The mobile transport device of claim 1, further comprising a remote monitoring unit.

25. A method for transporting insect larvae, the method comprising:
filling a first insect fattening container with a first insect larvae cohort and a fattening substrate;
inserting the filled first insect fattening container into a receiving section;
transporting the first insect larvae cohort from a first location to a second location; and
removing the first insect fattening container from the receiving section at the second location.

26. The method for transporting insect larvae of claim 25, further comprising:
cooling the first insect larvae cohort using a cooling unit;
maintaining the temperature during transport using the cooling unit; and
thawing and/or heating the first insect larvae cohort before or after removal of the insect larvae cohort from a mobile transport device.

27. The method for transporting insect larvae of claim 25, further comprising:
providing a first insect fattening container temperature signal representing a first insect fattening container temperature measurement value and providing a first insect fattening container humidity signal representing a first insect fattening container humidity measurement value from an activity sensor device to an electronic control unit;
determining an activity of the first insect larvae cohort received in the first insect fattening container based on the provided insect fattening container temperature signal and the provided insect fattening container humidity signal using the electronic control unit; and
adjusting control signals from the electronic control unit to a recirculation fan based on the determined activity of the first insect larvae cohort received in the first insect fattening container.

28. The method for transporting insect larvae of claim 27, further comprising:
adjusting control signals from the electronic control unit to a ventilation control unit based on the determined activity of the first insect larvae cohort received in the first insect fattening container for adjusting a first flow cross-section based on of the determined activity of the first insect larvae cohort received in the first insect fattening container.

29. The method for transporting insect larvae of claim 27, further comprising:
providing a first storage container temperature signal representing the first storage container temperature measurement value, providing a first interior humidity signal representing the first interior humidity measurement value, providing an interior temperature signal representing the first interior temperature measurement value, providing a CO2 concentration signal representing the CO2 concentration measurement value from the air sensor device to the electronic control unit;
determining a condition of air circulating in the housing based on the provided storage container temperature signal, the provided interior humidity signal, and the provided interior temperature signal; and
adjusting control signals from the electronic control unit to the storage container control unit based on the determined air condition.

30. The method for transporting insect larvae of claim 25, further comprising:
providing an exterior humidity signal representing a first exterior humidity measurement value and providing an exterior temperature signal representing a first exterior temperature measurement value from an air sensor device to the electronic control unit;
determining a condition of the ambient air based on the provided exterior humidity signal and the provided exterior temperature signal; and
adjusting control signals from the electronic control unit to a heating device.

31. The method for transporting insect larvae of claim 25, further comprising:
providing a CO2 concentration signal representing an measured CO2 concentration value from the air sensor device to the electronic control unit;
determining a CO2 concentration measurement value exceedance if the CO2 concentration measurement value exceeds a predetermined critical CO2 concentration value;
adjusting control signals from the electronic control unit to an fresh air fan if a CO2 concentration measurement value has been determined to be exceeded; and
adjusting control signals from the electronic control unit to an exhaust air fan if a CO2 concentration measurement value has been determined to be exceeded.

32. (canceled)
